(19) United States
(12) Patent Application Publication
(10) Pub. No.: US 2022/0396794 A1
(43) Pub. Date: Dec. 15, 2022

(54) APTAMERS AGAINST TRANSFERRIN RECEPTOR (TfR)

(71) Applicants: APTERNA LIMITED, London (GB); CITY OF HOPE, Duarte, CA (US)

(72) Inventors: Nagy HABIB, London (GB); John ROSSI, Duarte, CA (US); Sorah YOON, Duarte, CA (US); Piotr Marek Swidersk, San Dimas, CA (US)

(21) Appl. No.: 17/615,695

(22) PCT Filed: Jun. 3, 2020
(86) PCT No.: PCT/EP2020/065349

§ 371 Date: Dec. 1, 2021

Related U.S. Application Data
(60) Provisional application No. 62/856,791, filed on Jun. 4, 2019.

(51) Int. Cl. C12N 15/115; A61K 31/7068; A61P 35/04

(52) U.S. Cl. CPC C12N 15/115; A61K 31/7068; A61P 35/04; C12N 2310/16; C12N 2310/3519; C12N 2310/3517

(57) ABSTRACT

Methods of treating or preventing a disease or disorder are disclosed comprising administering to a subject in need thereof an effective amount of a nucleic acid compound comprising, or consisting of, a nucleic acid sequence capable of binding to a transferrin receptor (TfR) and an effective amount of an inhibitor of DNA synthesis. Also disclosed is a nucleic acid compound comprising, or consisting of, a nucleic acid sequence having at least 85% sequence identity to SEQ ID NO: 1, wherein said nucleic acid sequence is at least 30 nucleotides in length and at most 50 nucleotides in length, and wherein the nucleic acid sequence is capable of binding to a transferrin receptor (TfR).

### CROSS-REFERENCES TO RELATED APPLICATIONS

This application is a National Stage filing of International Patent Application No. PCT/EP2020/065349, filed Jun. 3, 2020, which claims the benefit of U.S. Provisional Application No. 62/856,791, filed Jun. 4, 2019, each of which are incorporated herein by reference in their entirety.

### REFERENCE TO A “SEQUENCE LISTING,” A TABLE, OR A COMPUTER PROGRAM LISTING APPENDIX SUBMITTED ON A COMPACT DISK

The instant application contains a Sequence Listing, which has been submitted electronically in ASCII format and is hereby incorporated by reference in its entirety. Said ASCII copy, created on Jul. 13, 2022, is named 048440-788N01US_SL_ST25 and is 10,780 bytes in size.

### FIELD OF THE INVENTION

The present invention relates to nucleic acid compounds, in particular ribonucleic acid compounds, capable of binding the transferrin receptor and compositions and methods using the same.

### BACKGROUND

Pancreatic ductal adenocarcinoma (PDAC) is one of the most aggressive malignant tumours with limited therapeutic efficacy and high mortality rates (Huguet et al 2009, Shaib et al 2006). Currently, first approach to cure PDAC can be achieved through surgical resection, however, the majority of PDAC patients are diagnosed in metastatic stages which is surgically unresectable. This type of advanced PDAC is a main causality of death in PDAC. PDAC preferentially metastasizes to the liver, which is the main cause of mortality related to advanced pancreatic cancer (Houg & Bijlsma, 2018)

The current standard treatments of advanced PDAC are limited to mono-chemotherapy; gemcitabine or combinational chemotherapy; gemcitabine combined with other chemotherapeutic agents such as 5FU, erlotinib, cisplatin, capecitabine, docetaxel, and oxaliplatin (Mohammad, 2018). However, combination chemotherapy does not show significantly statistical survival benefits for PDAC (Paulson et al 2013, hereby incorporated by reference in its entirety), and induction of chemotherapy prior to treatment with radiation with dose escalation results in severe side effects and the quality of life was not improved (Ma et al, 2018, hereby incorporated by reference in its entirety). To reduce side effects on healthy tissues, targeted delivery is preferred in the development of cancer therapeutics.

Targeted delivery of cancer therapeutics consists of two main approaches; passive targeting or active targeting. As passive targeting is solely depending on the enhanced permeability and retention (EPR) effects, less than 1% accumulates in xenografted tumour (Rosenblum et al, 2018, hereby incorporated by reference in its entirety). For specific homing and improving tumour localization by increasing targeting efficacy and retention at the target site throughout the uptake by target cells, the active targeting utilizes affinity ligands such as antibodies or aptamers.

Aptamers, sometimes described as chemical antibodies, are small single stranded RNA or DNA molecules that bind to their target through shape recognition (Stottenburg R et al., Biomol Eng. 2007 October; 24(4, hereby incorporated by reference in its entirety): 381-403). Aptamers comprise unique three-dimensional structures that are capable of specific molecular recognition of their cognate targets, and they display a number of advantages over antibodies, including their size, production process, increased stability and lack of immunogenicity.

The transferrin receptor (TfR) is a membrane glycoprotein expressed on the cellular surface which mediates cellular uptake of iron from the plasma glycoprotein transferrin. Transferrin binds to iron to create transferrin-iron complexes (Crichton & Charloteaux-Wauters, Eur J Biochem 1987; 164(3):485-506). These complexes bind to TfR and bound transferrin is internalised into cells via receptor-mediated endocytosis (Qian Z M et al., Pharmacol Rev. 2002, 54(4):561-587, hereby incorporated by reference in its entirety). Transferrin and iron are subsequently released in endosomes. TfR is typically expressed at low levels on a range of normal cells and is highly expressed on cells with high proliferation rates, including cancer cells (Daniels T R et al., Clin Immunol 2006 121: 144-158; Daniels T R et al., Clin Immunol 2006 121: 159-176, hereby incorporated by reference in its entirety). Thus, compounds capable of binding to TfR on the surface of TfR-expressing cells and internalising into the cell would be useful for targeted delivery of such compounds. Aptamers capable of binding TfR have been described in WO 2016/061386 and WO2019/033051, each hereby incorporated by reference in its entirety.

Aptamers that bind TfR could find use in providing targeted delivery of therapeutic payloads to cells throughout the body. Further mechanisms for targeted therapeutic delivery are needed.

### SUMMARY OF THE INVENTION

The present invention provides nucleic acid compounds, preferably ribonucleic acid compounds or deoxyribonucleic acid compounds, comprising a nucleic acid sequence which is capable of binding to a transferrin receptor (TfR). These find use for example in methods of medical treatment or prophylaxis and, in particular, in combination with inhibitors of DNA synthesis.

In a first aspect, the invention provides a nucleic acid compound for use in a method of medical treatment or prophylaxis, wherein the nucleic acid compound comprises, or consists of, a nucleic acid sequence capable of binding to a transferrin receptor (TfR), and wherein the method of medical treatment or prophylaxis comprises the step of administering the nucleic acid compound simultaneously or sequentially with an inhibitor of DNA synthesis

In a second aspect, the invention provides the use of a nucleic acid compound in the manufacture of a medicament for treating or preventing a disease or disorder, wherein the nucleic acid compound comprises, or consists of a nucleic acid sequence capable of binding to a transferrin receptor (TfR), and wherein the medicament is administered simultaneously or sequentially with an inhibitor of DNA synthesis.

In a third aspect, the invention provides a method of treating or preventing a disease or disorder, the method comprising administering to a subject in need thereof an effective amount of a pharmaceutical compound and an effective amount of an inhibitor of DNA synthesis, wherein said pharmaceutical compound comprises a nucleic acid compound comprising or consisting of a nucleic acid sequence capable of binding to a transferrin receptor (TfR).

In some embodiments, the nucleic acid sequence has least 85% sequence identity to any one of SEQ ID NO:1 to 6.

In some embodiments, the nucleic acid compound comprises or consists of a nucleic acid sequence that is any one of SEQ ID NOs: 1 to 6. In some embodiments, the nucleic acid sequence has a length of 22 nucleotides.

In some embodiments, the inhibitor of DNA synthesis is a nucleoside analogue. Preferably, the nucleoside analogue may be gemcitabine.

In some embodiments, the disease or disorder is cancer. In some embodiments, the disease or disorder is pancreatic cancer, preferably pancreatic ductal adenocarcinoma (PDAC).

In some embodiments, the nucleic acid compound further comprises a moiety attached to said nucleic acid sequence. The moiety may be therapeutic moiety, preferably an anticancer therapeutic moiety. In some embodiments, the therapeutic moiety is covalently attached to the nucleic acid sequence.

In some embodiments, the therapeutic moiety is a nucleic acid moiety, a peptide moiety or a small molecule drug moiety. In some embodiments, the therapeutic moiety is a miRNA, mRNA, saRNA or siRNA moiety. In particular embodiments, the therapeutic moiety is a C/EBPalpha saRNA moiety, a SIRT1 saRNA moiety, or a HNF saRNA moiety.

Also provided is a nucleic acid compound comprising, or consisting of, a nucleic acid sequence having at least 85% sequence identity to SEQ ID NO:5, wherein said nucleic acid sequence is at least 30 nucleotides in length and at most 50 nucleotides in length. The RNA sequence may preferably be capable of binding to a transferrin receptor (TfR). In some embodiments, the nucleic acid sequence has at least 85% sequence identity to SEQ ID NO: 2, 4 or 5. In some embodiments, the nucleic acid sequence is SEQ ID NO: 2, 4 or 5.

In some embodiments, the nucleic acid sequence has at least 85% sequence identity to SEQ ID NO:5, wherein said nucleic acid sequence is at least 30 nucleotides in length and at most 42 nucleotides in length, and wherein the nucleic acid sequence is capable of binding to a transferrin receptor (TfR).

In some embodiments, the nucleic acid sequence is 46 nucleotides in length and preferably has at least 85% sequence identity to SEQ ID NO:2. In some embodiments, the nucleic acid sequence has 100% sequence identity to SEQ ID NO:2.

In some embodiments, the nucleic acid sequence is 40 nucleotides in length and preferably has at least 85% sequence identity to SEQ ID NO:4. In some embodiments, the nucleic acid sequence has 100% sequence identity to SEQ ID NO:4.

In some embodiments, the nucleic acid sequence is 32 nucleotides in length and preferably has at least 85% sequence identity to SEQ ID NO:5. In some embodiments, the nucleic acid sequence has 100% sequence identity to SEQ ID NO:5.

In some embodiments, the nucleic acid compound is capable of binding to TfR on a cell surface. In some embodiments, the nucleic acid compound is capable of being internalised into a cell. In some embodiments, the nucleic acid compound has a 5′-GGG motif at the 5′ end of the nucleic acid sequence.

In some embodiments, a nucleic acid compound provided herein further comprises a compound moiety attached to the nucleic acid sequence. In some embodiments, the compound moiety is a therapeutic moiety or an imaging moiety. In some embodiments, the compound moiety is covalently attached to the nucleic acid sequence.

In some embodiments, the therapeutic moiety is a nucleic acid moiety, a peptide moiety or a small molecule drug moiety. In some embodiments, the therapeutic moiety is an activating nucleic acid moiety or an antisense nucleic acid moiety. In some embodiments, the therapeutic moiety is a miRNA, mRNA, saRNA or siRNA moiety. In some embodiments, the therapeutic moiety is an anticancer therapeutic moiety. In some embodiments, the therapeutic moiety is a C/EBPalpha saRNA moiety, a SIRT1 saRNA moiety, or a HNF saRNA moiety.

In some embodiments, the imaging moiety is a bioluminescent molecule, a photoactive molecule, a metal or a nanoparticle.

Also provided is a pharmaceutical composition comprising a nucleic acid compound according to the present invention. The composition may optionally comprise a pharmaceutically acceptable excipient. In some embodiments, the composition further comprises a therapeutic agent, optionally an anticancer agent. The anticancer agent may preferably be a DNA polymerase inhibitor e.g. gemcitabine.

The present invention also provides a method of delivering a compound moiety into a cell, the method comprising: (i) contacting a cell with a nucleic acid compound according to the present invention; and (ii) allowing said nucleic acid compound to bind to a transferrin receptor on said cell and pass into said cell thereby delivering said compound moiety into said cell.

Also provided is a method of delivering a compound into a cell, the method comprising: (i) contacting a cell with a compound and a nucleic acid compound according to the present invention; and (ii) allowing said nucleic acid compound to bind to a transferrin receptor on said cell and pass into said cell thereby delivering said compound into said cell. In some embodiments, the compound is a therapeutic agent or an imaging agent.

In another aspect the present invention provides a nucleic acid compound according to the present invention for use in a method of medical treatment or prophylaxis.

Also provided is the use of a nucleic acid compound according to the present invention in the manufacture of a medicament for treating or preventing a disease or disorder.

Also provided is a method treating or preventing a disease or disorder, the method comprising administering to a subject in need thereof an effective amount of a nucleic acid compound according to the present invention.

In some embodiments, the disease or disorder is cancer. In some embodiments, the method comprises administering an anticancer agent. In some embodiments, the disease or disorder is a metabolic disorder or a neurological disorder.

Also provided is a method of detecting a cell, the method comprising: (i) contacting a cell with a nucleic acid compound according to the present invention, wherein the nucleic acid compound comprises an imaging moiety; (ii) allowing the nucleic acid compound to bind to a transferrin receptor on said cell and pass into said cell; and (iii) detecting said imaging moiety thereby detecting said cell.

Also provided is a method of detecting a cell, the method comprising: (i) contacting a cell with an imaging agent and a nucleic acid compound according to the present invention; (ii) allowing the nucleic acid compound to bind to a transferrin receptor on said cell and allowing said imaging agent to pass into said cell; and (iii) detecting said imaging agent thereby detecting said cell.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIGS. 1A to 1I. Identification of an RNA aptamer against the human transferrin receptor (hTFRC). (a) The extracellular domain of hTFRC protein tagged with His6 was visualized using 10% SDS-PAGE and Coomassie stain; arrow indicates the protein at the expected size. M; marker, hTRFC; human transferrin receptor (10 μg/lane) (b) An RNA aptamer library pool was incubated with agarose beads to remove non-specific binders (1). The supernatant was incubated with His6-hTFRC for positive selection (2). After extraction of binders to hTFRC, RNA aptamers were amplified by PCR and in vitro transcription (3). After 9 rounds of SELEX, the aptamer sequence was identified. (C) The expected secondary structure of the full-length TR14 aptamer was predicted using Mfold. (D) TR14-hTFRC binding affinity and kinetics were determined using label-free biosensor assays and a Biacore T100 instrument. Positive response units (RUs) were observed following injection of hTFRC proteins. (E) To confirm the ability of the anti-hTfR TR14 aptamer to enter target cells, cell internalization assays were performed in PANC-1. 200 nM of Cy3-labeled RNA aptamer library or Cy3-labeled TR14 were incubated on live cells and visualized using confocal microscopy. Red: Cy3-labeled RNAs; blue: Hoechst 33342 for nuclei. Scale bar: 10 μm. (F) The binding of TR14 to target cells, PANC-1, was assessed by flow cytometry. (G) The uptake mechanism of TR14 was determined by small molecule inhibitors. Clathrin-mediated endocytosis (CME); chlorpromazine (CPZ), chloroquine (CQ), and dynasore. Clathrin independent endocytosis (CIE); genistein (GEZ, caveolae/lipid mediated endocytosis inhibitor) and cytochalasin D (Cyto D). (H) The competition assay of TR14 was assessed with transferrin and anti-TfR antibodies. (1) Subcellular co-localization of TR14 with early endosome, late endosome, and lysosomes was determined on live cells by confocal microscopy with Airyscan. The co-localized areas of TR14 with subcellular organs are presented in yellow where is indicated by white arrows. Red: Cy3-labeled RNAs; Green: GFP fused to Rab5a (early endosome marker), Rab7a (late endosome marker), or Lamp1 (lysosomal marker). Blue: Hoechst 33342 for nuclei. Scale bar: 5 μm.

FIGS. 2A to 2C. Truncation of an RNA aptamer against the human transferrin receptor 1 (hTfR1) (A) Description of in vitro transcription by T7 promoter to generate truncates of hTfR. “+1” marks the first nucleotides in the transcript during in vitro transcription. (B) The expected secondary structures of anti-transferrin receptor aptamers, TR14 truncates [S1 (46-nt), S2 (43-nt), ST1-1 (40-nt), ST1-2 (32-nt), ST1-3 (22-nt)], were predicted by NUPACK. Each color codes represent RNA nucleotides (C) Uptake of each TR14 truncates was confirmed by internalization assays with live cell imaging on two cancer cell lines; HepG2 and PANC-1 cells. 100 nM of each Cy3-labeled truncate was added to cancer cells and visualized using confocal microscopy. Red: Cy3-labeled RNAs; blue: Hoechst 33342 for nuclei. Scale bar: 10 μm.

FIGS. 3A to 3G. Internalization of TR14 and upregulation of C/EBPα in vitro. (A) PANC-1 cells were treated with cell control (CC), IRRE-TR14-CEBPA (irrelevant aptamer control), or TR14-CEBPA for 72 h. mRNA expression of C/EBPα and its downstream target p21 were measured using qPCR. (B) PANC-1 cells were treated with cell control (CC), IRRE-TR14 S2-CEBPA (irrelevant aptamer control), IRRE-TR14 ST1-3-CEBPA (irrelevant aptamer control), TR14 S2-CEBPA, or TR14 ST1-3-CEBPA for 72 h. mRNA expression of C/EBPα and its downstream target p21 were measured using qPCR. (C) PANC-1 cells were treated with cell control (CC), IRRE-tTR14-CEBPA (irrelevant aptamer control), tTR14-TC, tTR14-TCT, ortTR14-TCUT for 72 h. mRNA expression of C/EBPα and its downstream target p21 were measured using qPCR. One-way ANOVA test was used to determine statistical significance; *: P≤0.05, **: P≤0.01. (D) Inhibition of cell proliferation by IRRE-TR14-CEBPA or TR14-CEBPA was determined using MTS assay. (E) Inhibition of cell proliferation by cell control (CC), IRRE-TR14 S2-CEBPA (irrelevant aptamer control), IRRE-TR14 ST1-3-CEBPA (irrelevant aptamer control), TR14 S2-CEBPA, or TR14 ST1-3-CEBPA was determined using MTS assay. (F) Inhibition of cell proliferation by cell control (CC), IRRE-tTR14-CEBPA (irrelevant aptamer control), tTR14-TC, tTR14-TCT, or tTR14-TCUT for 72 h was determined using MTS assay. One-way ANOVA test was used to determine statistical significance; *: P≤0.05, **: P≤0.01. (G) To confirm the ability of the anti-hTFRC TR14 aptamer to enter target cells, cell internalization assays were performed in two cancer cell lines, HepG2 and PANC-1. 200 nM of Cy3-labeled RNA aptamer library or Cy3-labeled TR14 were incubated on live cells and visualized using confocal microscopy. Red: Cy3-labeled RNAs; blue: Hoechst 33342 for nuclei. Scale bar: 10 μm.

FIGS. 4A to 4B. Binding affinity of Truncated TR14 aptamer against hTfR1. (A) The truncates of TR14-hTfR1 binding affinity and kinetics were determined using label-free biosensor assays and a Biacore T100 instrument. Positive response units (RUs) were observed following injection of hTfR1 proteins on TR14 S1, TR14 S2, and TR14 ST1-3. (B) Cross-activity of TR14 S2 and TR14 ST1-3 was determined on selectively expressed of transferrin receptor 1 (TfR1) or transferrin receptor 2 (TfR2) on glioblastoma cells. U87MG cells are selectively expressed of TfR1, not TfR2. TB10 cells are selectively expressed of TfR2, not TfR1. Live cell imaging was performed by confocal microscopy. Red: Cy3-labeled RNAs; blue: Hoechst 33342 for nuclei. Scale bar: 20 μm.

FIGS. 5A to 5C. Establishment of liver-metastatic pancreatic cancer mouse model. PANC-Luc xenografted mice were tail-vein injected with PBS (CC), IRRE-TR14-CEBPA, TR14-CEBPA, IRRE-P19-CEBPA, or P19-CEBPA (1 nM). (A) Representative traceable tumor images show bioluminescence in the liver. (B) The expression of C/EBPα was determined using real-time qPCR. Data are presented as mean±standard deviation (SD); n=5-6. Unpaired t-test with Welch's correction was used to determine statistical significance; *: P≤0.05. (C) The expression of albumin, an indicator of liver function, was determined using qPCR. Data are presented as mean±SD; n=5-6. Unpaired t-test with Welch's correction was used to determine statistical significance.

FIGS. 6A to 6D. Anti-tumor effects of TR14- or P19-CEBPA-saRNAs in a liver-metastatic pancreatic cancer mouse model. PANC-Luc xenografted mice were tail-vein injected with PBS (CC), IRRE-TR14-CEBPA, TR14-CEBPA, IRRE-P19-CEBPA, or P19-CEBPA (1 nmol). (A and B) Liver tumor weight (A) and volume (B) were measured from liver biopsies. Data are presented as mean±standard deviation (SD). Student's t-test was used to determine statistical significance; *: P≤0.05, **: P≤0.01. (C) Tumor growth was monitored by evaluating the difference in bioluminescence before the first injection and one day after the last injection. Data are presented as the mean±SD. Student's t-test was used to determine statistical significance; *: P≤0.05. (D) Tumor growth was monitored by evaluating bioluminescence before the first injection and weekly throughout the 3-week treatment. Data are presented as the mean±SD. Student's t-test was used to determine statistical significance; *: P≤0.05, **: P≤0.01.

FIGS. 7A to 7D. Upregulation of C/EBPα and p21 in vitro and construction of conjugates with albumin tag (A) mRNA expression of C/EBPα and its downstream target p21 were measured using qPCR, after treatment. (B) Inhibition of cancer cell proliferation was measured by MTS assays. PANC-1 cells were treated with mock (CC), IRRE-TR14S2-CEBPA (irrelevant aptamer control), TR14-S2-CEBPA or TR14-ST1-3-CEBPA at 100 nM for 48 or 72 h. One-way ANOVA test was used to determine statistical significance; *: P≤0.05, **: P≤0.01. (C) mRNA expression of C/EBPα and its downstream target p21 were measured using qPCR after treatment of conjugates with Affinity tag. (D) Inhibition of cancer cell proliferation was measured by MTS assays after treatment of conjugates with Affinity tag. PANC-1 cells were treated with CC (mock), TC, TCT, TCUT for 48 or 72 h. One-way ANOVA test was used to determine statistical significance; *: P≤0.05, **: P≤0.01.

FIG. 8. Illustration of three conjugates with albumin affinity tag where relative position of different modules can be seen; TC, TCT, and TCUT. The TR14 ST1-3 (named tTR14) was modified at the 3′ end of saRNA sense strand with albumin-binding moiety (referred Affinity Tag).

FIGS. 9A to 9F. Anti-tumor effects of three conjugates in established liver-metastatic pancreatic cancer mouse model. PANC-Luc xenografted mice were tail-vein injected with PBS (CC), TC, TCT, TCUT at 1 nmol in combination with gemcitabine. (A) Representative traceable tumor images show bioluminescence in the liver-metastatic pancreatic cancer model. (B) Tumor growth was monitored by evaluating the tumor volume after treatment of each conjugates. Data are presented as the mean±SD. (C) Tumor growth was monitored by evaluating photon increase by administration of each conjugates. Data are presented as the mean±SD. Two-tailed Student's t-test was used to determine statistical significance. *: P≤0.05, **: P≤0.01. (D-F) The expression of C/EBPα in liver (D), brain (E) or albumin in liver (F) was determined using real-time qPCR. Data are presented as mean±standard deviation (SD); n=4-8. Unpaired t-test with Welch's correction was used to determine statistical significance. *: P≤0.05, **: P≤0.01.

FIG. 10. Alignment of TR14 full length and truncated sequences. 5′ poly-G is underlined.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & 20 Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, noncovalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogues or modified backbone residues or linkages, which are synthetic, naturally occurring, and non-naturally occurring, which have similar binding properties as the reference nucleic acid, and which are metabolized in a manner similar to the reference nucleotides. Examples of such analogues include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see

Eckstein, Oligonucleotides and Analogues: A Practical Approach, Oxford University Press); and peptide nucleic acid backbones and linkages. Other analogue nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogues can be made; alternatively, mixtures of different nucleic acid analogues, and mixtures of naturally occurring nucleic acids and analogues may be made. In embodiments, the internucleotide linkages in DNA are phosphodiester, phosphodiester derivatives, or a combination of both.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabelled or labelled as described below so that it's binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23:1087-1092; Kern (1997) Biotechniques 23:120-124; U.S. Pat. No. 5,143,854).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The term “aptamer” as provided herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, flexible structure, low immunogenicity, and versatile synthetic accessibility. To date, a variety of anti-cancer agents (e.g. chemotherapy drugs, toxins, and siRNAs) have been successfully delivered to cancer cells in vitro using aptamers.

An “antisense nucleic acid” as referred to herein is a nucleic acid (e.g. DNA or RNA molecule) that is complementary to at least a portion of a specific target nucleic acid (e.g. an mRNA translatable into a protein) and is capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone modified nucleotides.

In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem., 172:289 (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogues), short hairpin RNAs (shRNA), micro RNAs (miRNA), saRNAs (small activating RNAs) and small nucleolar RNAs (snoRNA) or certain of their derivatives or pre-cursors.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-30 nucleotides in length, e.g., 20, 21, 22, 23, 24, 25, 26, 27, 28, 29, or 30 nucleotides in length.

A “saRNA,” or “small activating RNA” as provided herein refers to a nucleic acid that forms a double stranded RNA, which double stranded RNA has the ability to increase or activate expression of a gene or target gene when expressed in the same cell as the gene or target gene. The complementary portions of the nucleic acid that hybridize to form the double stranded molecule typically have substantial or complete identity. In one embodiment, a saRNA is a nucleic acid that has substantial or complete identity to a target gene and forms a double stranded saRNA. Typically, the nucleic acid is at least about 15-50 nucleotides in length (e.g., each complementary sequence of the double stranded saRNA is 15-50 nucleotides in length, and the double stranded saRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “purified” denotes that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure.

The term “isolated” may also refer to a cell or sample cells. An isolated cell or sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample. In some embodiments, the cells are isolated through the use of a cell sorter. In some embodiments, antibodies against cell proteins are used to isolate cells.

As used herein, the term “conjugate” refers to the association between atoms or molecules. The association can be direct or indirect. For example, a conjugate between a nucleic acid (e.g., ribonucleic acid) and a compound moiety as provided herein can be direct, e.g., by covalent bond, or indirect, e.g., by non-covalent bond. Optionally, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. Thus, the nucleic acid acids can be attached to a compound moiety through its backbone. Optionally, the nucleic acid includes one or more reactive moieties, e.g., an amino acid reactive moiety, that facilitates the interaction of the nucleic acid with the compound moiety.

Useful reactive moieties or functional groups used for conjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.;

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g. phosphines) to form, for example, phosphate diester bonds; and

(n) sulfones, for example, vinyl sulfone.

The reactive functional groups can be chosen such that they do not participate in, or interfere with, the chemical stability of the proteins described herein. Byway of example, the nucleic acids can include a vinyl sulfone or other reactive moiety. Optionally, the nucleic acids can include a reactive moiety having the formula S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In some embodiments, about means the specified value.

The terms “protein”, “peptide”, and “polypeptide” are used interchangeably to denote an amino acid polymer or a set of two or more interacting or bound amino acid polymers. The terms apply to amino acid polymers in which one or more than one amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogues and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogues refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogues have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogues, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3)

Asparagine (N), Glutamine (Q); 4) Arginine (R), Lysine (K); 5) Isoleucine (I), Leucine (L),

Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

For specific proteins described herein (e.g., TfR), the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “TfR” as provided herein includes any of the transferrin receptor (TfR) protein naturally occurring forms, homologs or variants that maintain the activity of TfR (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the TfR protein is the protein as identified by the NCBI sequence reference GI: 189458817 (NCBI Reference Sequence: NP_003225.2; SEQ ID NO:13). In embodiments, the TfR protein is the protein as encoded by the nucleotide sequence identified by the NCBI sequence reference GI: 189458816 (NCBI Reference Sequence: NM_003234.3). In embodiments, the TfR protein is the protein as encoded by the nucleotide sequence identified by the NCBI sequence reference GI: 189458818 (NCBI Reference Sequence: NM_001128148.2). In embodiments, the TfR protein is the protein as identified by the NCBI sequence reference GI: 189458817 (NCBI Reference Sequence: NP_003225.2; SEQ ID NO:13), homolog or functional fragment thereof. In embodiments, the TfR protein is the protein as encoded by the nucleotide sequence identified by the NCBI sequence reference GI: 189458816 (NCBI Reference Sequence: NM_003234.3), homolog or functional fragment thereof. In embodiments, the TfR protein is the protein as encoded by the nucleotide sequence identified by the NCBI sequence reference GI: 189458818 (NCBI Reference Sequence: NM_001128148.2), homolog or functional fragment thereof. In embodiments, the TfR protein is encoded by a nucleic acid sequence corresponding to NCBI Gene ID: 7037.

The term “C/EBPα” or “C/EBPalpha” as provided herein includes any of the CCAAT (cytosine-cytosine-adenosine-adensoine-thymidine)/enhancer-binding protein alpha (C/EBPα) naturally occurring forms, homologs or variants that maintain the transcription factor activity of C/EBPalpha (e.g., within at least 50%, 80%, 90%, 95%, 96%, 97%, 98%, 99% or 100% activity compared to the native protein). In some embodiments, variants or homologs have at least 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the C/EBPalpha protein is the protein as identified by the NCBI sequence reference GI: 28872794 (NP_004355.2), GI: 551894998 (NP_001272758.1), GI: 566559992 (NP_001274353.1), or GI: 566559994 (NP_001274364.1), or homolog or functional fragment thereof. In embodiments, the C/EBPalpha protein is encoded by a nucleic acid sequence corresponding to Gene ID: 1050.

The term “sirtuin” refers to one or more of the sirtuin class of proteins that possess either mono-ADP-ribosyltransferase or deacetylase activity (including deacetylase, desuccinylase, demalonylase, demyristoylase and depalmitoylase activity). They are dependent on nicotine adenine dinucleotide (NAD) and have been implicated in regulating ageing mechanisms, responses to stress, and disorders such as cancer and diabetes (see e.g. North and Verdin, Genome Biol. 2004, 5(5): 224; Preyat and Leo, J. Leukoc. Biol. 2013, 93(5): 669-680; and Satoh A et al., J Neurosci. 2010, 30(30): 10220-10232, which are all hereby incorporated by reference in their entirety). The human genome encodes seven sirtuin genes: SIRT1 to SIRT7.

“SIRT1”, “SIRT2”, “SIRT3”, “SIRT4”, “SIRT5”, “SIRT6” and “SIRT7” refer to the human Sirtuin genes that encode the Sirt1 to Sirt7 proteins, respectively, including homologs and variants thereof that produce a protein product that maintains the deacetylase activity of one or more of Sirt1-Sirt7 (e.g., within at least 50%, 80%, 90%, 95%, 96%, 97%, 98%, 99% or 100% activity compared to the native protein). The terms “Sirt1”, “Sirt2”, “Sirt3”, “Sirt4”, “Sirt5”, “Sirt6”, and “Sirt7” as provided herein include any naturally occurring forms, homologs or variants that maintain the deacetylase activity of said sirtuin proteins (e.g., within at least 50%, 80%, 90%, 95%, 96%, 97%, 98%, 99% or 100% activity compared to the native protein). In some embodiments, variants or homologs have at least 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In some embodiments, human Sirt1 protein is the protein as identified by the NCBI Reference Sequence: NP_036370.2. In some embodiments, the Sirt1 protein is encoded by a nucleic acid sequence corresponding to Gene ID: 23411. Human Sirt2 may be the protein as identified by GenBank reference AAK51133.1, and may be encoded by a nucleic acid sequence corresponding to Gene ID: 22933. Human Sirt3 may be the protein as identified by GenBank reference AAD40851.1, and may be encoded by a nucleic acid sequence corresponding to Gene ID: 23410. Human Sirt4 may be the protein as identified by NCBI reference NP_036372.1, and may be encoded by a nucleic acid sequence corresponding to Gene ID: 23409. Human Sirt5 may be the protein as identified by GenBank reference AAD40853.1, and may be encoded by a nucleic acid sequence corresponding to Gene ID: 23408. Human Sirt6 may be the protein as identified by GenBank reference CAG33481.1, and may be encoded by a nucleic acid sequence corresponding to Gene ID: 51548. Human Sirt7 may be the protein as identified by NCBI reference NP_057622.1, and may be encoded by a nucleic acid sequence corresponding to Gene ID: 51547.

The term “HNF” refers to one or more hepatocyte nuclear factors. Hepatocyte nuclear factors are a group of transcription factors expressed predominantly in the liver which regulate gene expression. HNF may refer to hepatocyte nuclear factor 4 (HNF4), a nuclear receptor protein expressed mostly in the liver, gut, kidney and pancreatic beta cells. There are two isoforms of human HNF4: HNF4a and HNF4γ expressed by the genes HNF4A and HNF4G, respectively. Human HNF4a and/or HNF4A may be the protein/gene as identified by Uniprot P41235, which also describes at least 7 isoforms of HNF4α that are produced by alternative promoter usage and alternative splicing. Human HNF4γ and/or HNF4G may be the protein/gene as identified by Uniprot Q14541, which also describes two isoforms of HNF4γ produced by alternative splicing. Mutations in or variations of the HNF4A gene have been linked to metabolic disorders including maturity-onset diabetes of the young 1 (MODY1; see e.g. Bulman M P et al., Diabetologia. 1997, 40(7):859-62), non-insulin dependent diabetes mellitus (NIDDM; see e.g. Hani E H et al., J Clin Invest. 1998, 101(3):521-6), and Fanconi renotubular syndrome 4 with maturity-onset diabetes of the young (FRTS4; see e.g. Hamilton A J et al., J Med Genet. 2014, 51(3):165-9). The terms “HNF4α” and “HNF4γ” include any naturally occurring forms, homologs or variants that maintain the activity of HNF4α or HNF4γ (e.g., within at least 50%, 80%, 90%, 95%, 96%, 97%, 98%, 99% or 100% activity compared to the native protein). In some embodiments, variants or homologs have at least 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. The terms “HNF4A” and “HNF4G” include genes as well as homologs and variants thereof that produce a protein product that maintains the activity of one or more of HNF4α and/or HNF4γ (e.g., within at least 50%, 80%, 90%, 95%, 96%, 97%, 98%, 99% or 100% activity compared to the native protein).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “blood-brain barrier” refers to a highly selective semipermeable membrane barrier that separates the circulating blood from the brain and extracellular fluid in the central nervous system. The barrier provides tight regulation of the movement of ions, molecules and cells between the blood and the brain, see e.g. Daneman and Prat, Cold Spring Harb Perspect Biol. 2015; 7(1):a020412. Many therapeutic molecules are generally excluded from transport from blood to brain due to their negligible permeability over the brain capillary endothelial wall.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anticancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analogue (e.g., methotrexate), or pyrimidine analogues (e.g., fluorouracil, floxouridine, Cytarabine), purine analogues (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), anti tumour antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), or adrenocortical suppressant (e.g., mitotane, aminoglutethimide), epipodophyllotoxins (e.g., etoposide).

Further examples of anti-cancer agents include, but are not limited to, antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-I receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein kinase C inhibitors, protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the nucleic acid compound described herein can be co-administered with or covalently attached to conventional immunotherapeutic agents including, but not limited to, immunostimulants (e.g., Bacillus Calmette-Guerin (BCG), levamisole, interleukin-2, alphainterferon, etc.), monoclonal antibodies (e.g., anti-CD20, anti-HER2, anti-CD52, anti-HLA-DR, anti-PD-1 and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the nucleic acid compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm ¹⁵³Sm, ¹⁶⁶Ho, ¹⁷⁷Lu ¹⁸⁶Re, ¹⁸⁸Re, ²¹¹At, and ²¹²Bi, optionally conjugated to antibodies directed against tumour antigens.

The term “sample” includes sections of tissues such as biopsy and autopsy samples, and frozen sections taken for histological purposes. Such samples include blood and blood fractions or products (e.g., bone marrow, serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyse data based on comparisons to control values. Controls are also valuable for determining the significance of data. For example, if values for a given parameter are widely variant in controls, variation in test samples will not be considered as significant.

“Disease” or “condition” refer to a state of being or health status of a patient or subject capable of being treated with a compound, pharmaceutical composition, or method provided herein. In embodiments, the disease is cancer (e.g. liver cancer, pancreatic cancer, pancreatic liver metastases, brain cancer, prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma), an infectious disease (e.g., HN infection), an inflammatory disease (e.g., rheumatoid arthritis) or a metabolic disease (e.g., diabetes). In embodiments, the disease is a disease related to (e.g. caused by) an aberrant activity of TfR, TfR phosphorylation, or TfR pathway activity, or pathway activated by TfR. In some embodiments, the disease is cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma).

As used herein, the term “cancer” refers to all types of cancer, neoplasm or malignant tumours found in mammals, including leukaemia, lymphoma, carcinomas and sarcomas.

Exemplary cancers that may be treated with a compound, pharmaceutical composition, or method provided herein include pancreatic cancer, liver cancer (e.g. hepatocellular carcinoma), pancreatic liver metastases, lymphoma, sarcoma, bladder cancer, bone cancer, brain cancer (e.g. brain tumour, medulloblastoma, glioblastoma, glioblastoma multiforme), cervical cancer, colon cancer, oesophageal cancer, gastric cancer, head and neck cancer, kidney cancer, myeloma, thyroid cancer, leukaemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, lung cancer (e.g. non-small cell lung carcinoma, squamous cell lung carcinoma, adenocarcinoma, large cell lung carcinoma, small cell lung carcinoma, carcinoid, sarcoma), glioma, neuroblastoma, melanoma, castration-resistant prostate cancer, squamous cell carcinoma (e.g., head, neck, or esophagus), colorectal cancer, acute myeloid leukaemia, B cell lymphoma, multiple myeloma, Hodgkin's Disease, Non-Hodgkin's Lymphoma, rhabdomyosarcoma, mesothelioma, endometrial cancer, thrombocytosis, Waldenstrom macroglobulinemia (WM), insulanoma, malignant carcinoid, premalignant skin lesions, testicular cancer, malignant hypercalcemia, adrenal cortical cancer, neoplasms of the endocrine or exocrine pancreas, medullary thyroid cancer, medullary thyroid carcinoma, papillary thyroid cancer, Paget's Disease of the Nipple, Phyllodes Tumors, Lobular Carcinoma, Ductal Carcinoma, cancer of the pancreatic stellate cells, or cancer of the hepatic stellate cells. Additional examples include cancer of the endocrine system, brain, breast, bone, cervix, colon, head & neck, oesophagus, liver, kidney, lung, non-small cell lung, ovary, stomach, mouth, skin, uterus, endometrium, pancreas, thyroid, bladder, prostate, testicle or genitourinary tract.

The term “leukaemia” refers broadly to progressive, malignant diseases of the blood-forming organs and is generally characterized by a distorted proliferation and development of leukocytes and their precursors in the blood and bone marrow. Leukaemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukemic leukaemia, a leukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairycell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, multiple myeloma, plasmacytic leukaemia, promyelocytic leukaemia, Rieder cell leukaemia, Schilling's leukaemia, stem cell leukaemia, subleukemic leukaemia, or undifferentiated cell leukaemia.

The term “sarcoma” generally refers to a tumour which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumour arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumour, e.g., primary breast cancer. Some cancer cells in the primary tumour or originating site acquire the ability to penetrate and infiltrate surrounding normal tissue in the local area and/or the ability to penetrate the walls of the lymphatic system or vascular system circulating through the system to other sites and tissues in the body. A second clinically detectable tumour formed from cancer cells of a primary tumour is referred to as a metastatic or secondary tumour. When cancer cells metastasize, the metastatic tumour and its cells are presumed to be similar to those of the original tumour. Thus, if lung cancer metastasizes to the breast, the secondary tumour at the site of the breast consists of abnormal lung cells and not abnormal breast cells. The secondary tumour in the breast is referred to a metastatic lung cancer. Thus, the phrase metastatic cancer refers to a disease in which a subject has or had a primary tumour and has one or more secondary tumours. The phrases non-metastatic cancer or subjects with cancer that is not metastatic refers to diseases in which subjects have a primary tumour but not one or more secondary tumours. For example, metastatic lung cancer refers to a disease in a subject with or with a history of a primary lung tumour and with one or more secondary tumours at a second location or multiple locations, e.g., in the breast.

The term “associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma)) means that the disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma) or viral disease (e.g., HN infection associated disease)) is caused by (in whole or in part), or a symptom of the disease is caused by (in whole or in part) the substance or substance activity or function.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. Contacting may include allowing two species to react, interact, or physically touch, wherein the two species may be a nucleic acid compound as described herein and a cell (e.g., cancer cell). Simultaneous administration refers to administration of the agents together, for example as a pharmaceutical composition containing the agents (i.e. a combined preparation), or immediately after each other and optionally via the same route of administration, e.g. to the same artery, vein or other blood vessel. In particular embodiments, the nucleic acid sequence capable of binding to a transferrin receptor (TfR) and the inhibitor of DNA synthesis may be administered simultaneously in a combined preparation. In certain embodiments upon simultaneous administration the two or more of the agents may be administered via different routes of administration. In some embodiments simultaneous administration refers to administration at the same time, or within e.g. 1 hr, 2 hrs, 3 hrs, 4 hrs, 5 hrs, 6 hrs, 8 hrs, 12 hrs, 24 hrs, 36 hrs or 48 hrs.

Sequential administration refers to administration of one or more of the agents followed after a given time interval by separate administration of another of the agents. It is not required that the two agents are administered by the same route, although this is the case in some embodiments. The time interval may be any time interval, including hours, days, weeks, months, or years. In some embodiments sequential administration refers to administrations separated by a time interval of one of at least 10 min, 30 min, 1 hr, 6 hrs, 8 hrs, 12 hrs, 24 hrs, 36 hrs, 48 hrs, 3 days, 4 days, 5 days, 6 days, 1 week, 2 weeks, 3 weeks, 1 month, 6 weeks, 2 months, 3 months, 4 months, 5 months or 6 months.

### Nucleic Acid Compounds

The present invention provides nucleic acid compounds that are inter alia capable of binding a transferrin receptor (TfR). In preferred embodiments, the TfR is on a cell and, in some cases, the nucleic acid compounds are internalised into the cell.

TfR is expressed at low levels on normal cells. Cells with high-proliferation rates, such as activated immune cells and cancers, present upregulated expression of TfR. The nucleic acid compounds of the present invention thus provide a mechanism to target a broad variety of cells via TfR binding.

In various embodiments, the nucleic acid compounds provided herein comprise a payload, such as a therapeutic or diagnostic molecule, and thus facilitate targeted delivery of the payload to TfR-expressing cells. The nucleic acid compounds and the payload may be internalised into TfR-expressing cells, thus providing an efficient mechanism for targeted intracellular delivery.

WO 2016/061386 describes nucleic acid compounds that are capable of binding TfR. The nucleic acid compounds in WO 2016/061386 comprise RNA sequences having at least 30 nucleotides and are exemplified by compounds comprising RNA sequences that are 87 or 43 nucleotides in length.

The three-dimensional structure of a nucleic acid compound, e.g. an aptamer, is essential for determining binding affinity and specificity. Thus, one cannot truncate a nucleic acid compound with the absolute expectation that it will retain its ability to bind the same target. Predicting functional truncated aptamer sequences is not a trivial exercise.

In addition, thanks to their reduced size, the nucleic acid compounds described herein are capable of crossing the blood-brain barrier (BBB) and delivering therapeutic or diagnostic payloads to TfR-expressing cell targets in the brain.

Thus, the nucleic acid compounds of the present invention provide highly specific and efficient means for targeted delivery of payloads to a range of cell types in multiple animal species, as shown herein. The present invention also provides a valuable mechanism to overcome the almost impermeable, highly-selective and well-coordinated BBB and achieve delivery of therapeutic and imaging agents to the brain.

Provided herein are novel nucleic acid compounds. The nucleic acid compounds of the invention may comprise ribonucleic acids and/or deoxyribonucleic acids. The nucleic acid compounds comprise a nucleic acid sequence, which may be a DNA sequence or an RNA sequence. In some embodiments, the nucleic acid compound is a ribonucleic acid compound comprising an RNA sequence.

In some aspects, the present invention provides a nucleic acid compound comprising, or consisting of, a nucleic acid sequence having at least 85% sequence identity to SEQ ID NO:5, and wherein the nucleic acid sequence is at least 30 nucleotides in length and at most 50 nucleotides in length. In any embodiment provided herein, the nucleic acid compound may be a ribonucleic acid compound.

In some embodiments, the nucleic acid compound does not consist of a nucleic acid sequence according to SEQ ID NO:3. In some embodiments, the nucleic acid compound does not consist of a nucleic acid sequence according to SEQ ID NO:6. In some embodiments, the nucleic acid compound does not comprise or consist of a nucleic acid sequence according to SEQ ID NO:1.

In some embodiments, the nucleic acid compound comprises, or consists of a nucleic acid sequence that is at least 30, 31, 32, 33, 34, 35, 36, 37, 38, 39, 40, 41, 42, 43, 44, 45, 46, 47, 48, 49 or at least 50 nucleotides in length. In some embodiments, the nucleic acid compound comprises, or consists of a nucleic acid sequence that is at most 30, 31, 32, 33, 34, 35, 36, 37, 38, 39, 40, 41, 42, 43, 44, 45, 46, 47, 48, 49 or at most 50 nucleotides in length. In some embodiments, the nucleic acid compound comprises, or consists of a nucleic acid sequence that is at least 30 and most 50 nucleotides in length.

In some embodiments, the nucleic acid sequence is 30, 31, 32, 33, 34, 35, 36, 37, 38, 39, 40, 41, or 42 nucleotides in length. In some embodiments, the nucleic acid sequence is 44, 45, 46, 47, 48, 49 or 50 nucleotides in length. In some embodiments, the nucleic acid sequence is less than 43 nucleotides in length. In some embodiments, the nucleic acid sequence is more than 43 nucleotides in length.

In some embodiments, the nucleic acid compound comprises, or consists of a nucleic acid sequence that is at least 30 and at most 46 nucleotides in length. In some embodiments, the nucleic acid compound comprises, or consists of a nucleic acid sequence that is at least 44 and at most 50 nucleotides in length. In some embodiments, the nucleic acid compound comprises, or consists of a nucleic acid sequence that is at least 44 and at most 46 nucleotides in length. In some embodiments, the nucleic acid compound comprises, or consists of a nucleic acid sequence having a length of 46 nucleotides. In some embodiments, the nucleic acid compound comprises, or consists of a nucleic acid sequence that is at least 30 and at most 40 nucleotides in length. In some embodiments, the nucleic acid compound comprises, or consists of a nucleic acid sequence that is at least 32 and at most 40 nucleotides in length. In some embodiments, the nucleic acid compound comprises, or consists of a nucleic acid sequence that is at least 35 and at most 40 nucleotides in length. In some embodiments, the nucleic acid compound comprises, or consists of a nucleic acid sequence that is at least 30 and at most 35 nucleotides in length. In some embodiments, the nucleic acid compound comprises, or consists of a nucleic acid sequence that is at least 30 and at most 32 nucleotides in length. In some embodiments, the nucleic acid compound comprises, or consists of a nucleic acid sequence having a length of 40 nucleotides. In some embodiments, the nucleic acid compound comprises, or consists of a nucleic acid sequence having a length of 32 nucleotides.

In some embodiments, the nucleic acid sequence is from 23 to 50, from 23 to 46, from 23 to 42, from 23 to 32, from 30 to 50, from 30 to 46, from 30 to 42, from 30 to 35, from 32 to 42, from 32 to 46, from 32 to 50, from 40 to 42, from 44 to 46, from 44 to 50, or from 46 to 50 nucleotides in length.

In some embodiments, the nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO:5. In some embodiments, the nucleic acid sequence has 85%, 86%, 87%, 88%, 89%, 90%, 91%, 92%, 93%, 94%, 95%, 96%, 97%, 98%, 99% or 100% sequence identity to SEQ ID NO:5. In some embodiments, the nucleic acid sequence consists of SEQ ID NO:5.

In some embodiments, the nucleic acid sequence hybridises with a SEQ ID NO:5. In some embodiments, the nucleic acid sequence hybridises with a sequence which has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO:5. In some embodiments, the nucleic acid sequence hybridises with a sequence which has 85%, 86%, 87%, 88%, 89%, 90%, 91%, 92%, 93%, 94%, 95%, 96%, 97%, 98%, 99% or 100% sequence identity to SEQ ID NO:5.

In some embodiments, the nucleic acid sequence has at least 85% sequence identity to any of SEQ ID NOs 2, 4 or 5.

In some embodiments, the nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO:2. In some embodiments, the nucleic acid sequence has 85%, 86%, 87%, 88%, 89%, 90%, 91%, 92%, 93%, 94%, 95%, 96%, 97%, 98%, 99% or 100% sequence identity to SEQ ID NO:2. In some embodiments, the nucleic acid sequence is 46 nucleotides in length, and preferably has at least 85% sequence identity to SEQ ID NO:2. In some embodiments, the nucleic acid sequence consists of SEQ ID NO:2.

In some embodiments, the nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO:4. In some embodiments, the nucleic acid sequence has 85%, 86%, 87%, 88%, 89%, 90%, 91%, 92%, 93%, 94%, 95%, 96%, 97%, 98%, 99% or 100% sequence identity to SEQ ID NO:4. In some embodiments, the nucleic acid sequence is 40 nucleotides in length, and preferably has at least 85% sequence identity to SEQ ID NO:4. In some embodiments, the nucleic acid sequence consists of SEQ ID NO:4.

In some embodiments, the nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO:5. In some embodiments, the nucleic acid sequence has 85%, 86%, 87%, 88%, 89%, 90%, 91%, 92%, 93%, 94%, 95%, 96%, 97%, 98%, 99% or 100% sequence identity to SEQ ID NO:5. In some embodiments, the nucleic acid sequence is 32 nucleotides in length, and preferably has at least 85% sequence identity to SEQ ID NO:5. In some embodiments, the nucleic acid sequence consists of SEQ ID NO:5.

In some embodiments, the nucleic acid sequence hybridises with any of SEQ ID NOs 2, 4 or 5. In some embodiments, the nucleic acid sequence hybridises with a sequence which has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to any of SEQ ID NOs:2, 4 or 5.

In some embodiments, the nucleic acid compound may have one or more substitutions relative to a reference sequence, i.e. any of SEQ ID NOs 1, 2, 4 or 5. In some embodiments, the nucleic acid compound comprises, or consists of a nucleic acid sequence having at most 1, 2, 3, 4 or 5 substitutions relative to a reference sequence.

In some embodiments of any of the aspects of the invention, the nucleic acid sequence is capable of binding to a transferrin receptor (TfR). In some embodiments, the nucleic acid sequence binds to a transferrin receptor (TfR). In some embodiments, the TfR is on a cell surface. In some embodiments, the nucleic acid compound is capable of being internalised into a cell. In some cases, the cell is a TfR-expressing cell.

In some embodiments, the nucleic acid compound has a 5′-GGG motif at the 5′ end of the nucleic acid sequence. Without wishing to be bound by theory, a 5′-GGG motif at the 5′ end of the nucleic acid sequence may increase the extent to which the nucleic acid compound is internalised into a cell. A 5′-GGG motif may be sufficient, if not necessary, for internalisation into a cell. In some embodiments, the nucleic acid compound does not have a 5′-GGG motif at the 5′ end of the nucleic acid sequence.

In some embodiments, the nucleic acid sequence has an equilibrium dissociation constant (K^{D}) for TfR of less than about 1×10⁻⁸, 1×10⁻⁹, 1×10⁻¹⁰, 1×10⁻¹¹, or less than 1×10⁻¹² M. In some embodiments, the nucleic acid sequence has K^{D} for TfR of between 1×10⁻⁸ and 1×10⁻¹³ M, between 1×10⁻⁹ and 1×10⁻¹³ M, between 1×10⁻¹⁰ and 1×10⁻¹³ M, between 1×10⁻¹¹ and 1×10⁻¹³ M, or between 1×10⁻¹² and 1×10⁻¹³ M. In some embodiments, the nucleic acid sequence has a K^{D} for TfR of between 1 and 5×10⁻¹⁰ M, of between 1 and 5×10⁻¹¹ M, or of between 5×10⁻¹³ and 1×10⁻¹² M. In some embodiments, the nucleic acid sequence has a K^{D} for TfR of between 1×10⁻⁹ and 1×10⁻¹⁰ M, 1×10⁻¹⁰ and 1×10⁻¹¹ M, or of 1×10⁻¹² and 1×10⁻¹³ M. In some embodiments, the nucleic acid sequence has a higher affinity for TfR than TR14.

In some embodiments, the nucleic acid compound comprises or consists of a nucleic acid sequence having at least 85% sequence identity to any one of SEQ ID NOs 14 to 16. In some embodiments, the nucleic acid sequence comprises or consists of any one of SEQ ID NOs 14 to 16.

In some aspects, the invention provides a nucleic acid compound comprising, or consisting of, a nucleic acid sequence having at least 85% sequence identity to SEQ ID NO:2, and wherein the nucleic acid sequence is capable of binding to a transferrin receptor (TfR).

In some aspects, the invention provides a nucleic acid compound comprising, or consisting of, a nucleic acid sequence having at least 85% sequence identity to SEQ ID NO:4, and wherein the nucleic acid sequence is capable of binding to a transferrin receptor (TfR).

In some aspects, the invention provides a nucleic acid compound comprising, or consisting of, a nucleic acid sequence having at least 85% sequence identity to SEQ ID NO:5, and wherein the nucleic acid sequence is capable of binding to a transferrin receptor (TfR).

In a first particular aspect, the invention provides a nucleic acid compound comprising, or consisting of, a nucleic acid sequence having at least 85% sequence identity to SEQ ID NO:5, wherein said nucleic acid sequence is at least 30 nucleotides in length and at most 42 nucleotides in length, and wherein the nucleic acid sequence is capable of binding to a transferrin receptor (TfR).

In some embodiments of the first particular aspect, the nucleic acid sequence is at least 31 nucleotides in length and at most 42 nucleotides in length. In some embodiments of the first particular aspect, the nucleic acid sequence is at least 32 nucleotides in length and at most 42 nucleotides in length. In some embodiments of the first particular aspect, the nucleic acid sequence is at least 31 nucleotides in length and at most 41 nucleotides in length. In some embodiments of the first particular aspect, the nucleic acid sequence is at least 32 nucleotides in length and at most 41 nucleotides in length. In some embodiments of the first particular aspect, the nucleic acid sequence is at least 30 nucleotides in length and at most 40 nucleotides in length. In some embodiments of the first particular aspect, the nucleic acid sequence is at least 31 nucleotides in length and at most 40 nucleotides in length.

In some embodiments of the first particular aspect, the nucleic acid sequence is 30, 31, 32, 33, 34, 35, 36, 37, 38, 39, 40, 41 or 42 nucleotides in length.

In some embodiments of the first particular aspect, the nucleic acid compound comprises, or consists of, a nucleic acid sequence having at least 85%, at least 90%, at least 95%, at least 96%, at least 97%, at least 98%, at least 99% or 100% sequence identity to SEQ ID NO:5. In some embodiments of the first particular aspect, the nucleic acid compound comprises, or consists of, a nucleic acid sequence having at least 85%, at least 90%, at least 95%, at least 96%, at least 97%, at least 98%, at least 99% or 100% sequence identity to SEQ ID NO:4.

In some embodiments of the first particular aspect, the nucleic acid sequence is capable of binding to both TfR1 and TfR2.

In some embodiments of the first particular aspect, the nucleic acid compound further comprises a compound moiety attached to the nucleic acid sequence. In some embodiments, the moiety is a therapeutic moiety, e.g. an anticancer therapeutic moiety. In some embodiments, the moiety is covalently attached to said nucleic acid sequence. In some embodiments, the moiety is covalently attached to the nucleic acid sequence by a linker. In some embodiments, the moiety is a saRNA selected from SEQ ID NO:7 and SEQ ID NO:9.

In some embodiments of the first particular aspect, the nucleic acid compound further comprises an albumin tag attached to the nucleic acid sequence.

In a second particular aspect, the invention provides a nucleic acid compound comprising, or consisting of, a nucleic acid sequence having at least 85% sequence identity to SEQ ID NO:2, wherein said nucleic acid sequence is at least 44 nucleotides in length and at most 50 nucleotides in length, and wherein the nucleic acid sequence is capable of binding to a transferrin receptor (TfR).

In some embodiments of the second particular aspect, the nucleic acid sequence is at least 44 nucleotides in length and at most 50 nucleotides in length. In some embodiments of the second particular aspect, the nucleic acid sequence is at least 45 nucleotides in length and at most 50 nucleotides in length. In some embodiments of the second particular aspect, the nucleic acid sequence is at least 46 nucleotides in length and at most 50 nucleotides in length. In some embodiments of the second particular aspect, the nucleic acid sequence is at least 44 nucleotides in length and at most 49 nucleotides in length. In some embodiments of the second particular aspect, the nucleic acid sequence is at least 45 nucleotides in length and at most 49 nucleotides in length. In some embodiments of the second particular aspect, the nucleic acid sequence is at least 46 nucleotides in length and at most 49 nucleotides in length. In some embodiments of the second particular aspect, the nucleic acid sequence is at least 44 nucleotides in length and at most 48 nucleotides in length. In some embodiments of the second particular aspect, the nucleic acid sequence is at least 45 nucleotides in length and at most 48 nucleotides in length. In some embodiments of the second particular aspect, the nucleic acid sequence is at least 46 nucleotides in length and at most 48 nucleotides in length. In some embodiments of the second particular aspect, the nucleic acid sequence is at least 44 nucleotides in length and at most 47 nucleotides in length. In some embodiments of the second particular aspect, the nucleic acid sequence is at least 45 nucleotides in length and at most 47 nucleotides in length. In some embodiments of the second particular aspect, the nucleic acid sequence is at least 46 nucleotides in length and at most 47 nucleotides in length. In some embodiments of the second particular aspect, the nucleic acid sequence is at least 44 nucleotides in length and at most 46 nucleotides in length. In some embodiments of the second particular aspect, the nucleic acid sequence is at least 45 nucleotides in length and at most 46 nucleotides in length.

In some embodiments of the second particular aspect, the nucleic acid sequence is 44, 45, 46, 47, 48, 49 or 50 nucleotides in length.

In some embodiments of the second particular aspect, the nucleic acid compound comprises, or consists of, a nucleic acid sequence having at least 85%, at least 90%, at least 95%, at least 96%, at least 97%, at least 98%, at least 99% or 100% sequence identity to SEQ ID NO:2.

In some embodiments of the second particular aspect, the nucleic acid compound further comprises a compound moiety attached to the nucleic acid sequence. In some embodiments, the moiety is a therapeutic moiety, e.g. an anticancer therapeutic moiety. In some embodiments, the moiety is covalently attached to the nucleic acid sequence. In some embodiments, the moiety is covalently attached to the nucleic acid sequence by a linker. In some embodiments, the moiety is a saRNA selected from SEQ ID NO:7 and SEQ ID NO:9.

In some embodiments of the second particular aspect, the nucleic acid compound further comprises an albumin tag attached to the nucleic acid sequence.

The disclosure also relates to a pharmaceutical composition comprising a nucleic acid compound according to the first or second particular aspect, optionally comprising a pharmaceutically acceptable excipient.

The disclosure also relates to a method of delivering a compound moiety into a cell, the method comprising:

i. contacting a cell with a nucleic acid compound according to the first or second particular aspect, or with a composition comprising a nucleic acid compound according to the first or second particular aspect; and

ii. allowing said nucleic acid compound to bind to a transferrin receptor on said cell and pass into said cell thereby delivering said compound moiety into said cell.

The disclosure also relates to a method of delivering a compound into a cell, the method comprising:

i. contacting a cell with a compound and the nucleic acid compound according to the first or second particular aspect

ii. allowing said nucleic acid compound to bind to a transferrin receptor on said cell and pass into said cell thereby delivering said compound into said cell.

Also provided is a nucleic acid compound according to the first or second particular aspect or a composition comprising a nucleic acid compound according to the first or second particular aspect, for use in a method of medical treatment or prophylaxis for a disease or disorder. Also provided is the use of a nucleic acid compound according to the first or second particular aspect, or a composition comprising a nucleic acid compound according to the first or second particular aspect, in the manufacture of a medicament for treating or preventing a disease or disorder. Also provided is a method of treating or preventing a disease or disorder, the method comprising administering to a subject in need thereof an effective amount of a nucleic acid compound according to the first or second particular aspect, or a composition comprising a nucleic acid compound according to the first or second particular aspect. In these provisions, the disease or disorder may be cancer, and in a particular may be HDAC.

Combination Therapy with Inhibitors of DNA Synthesis

The invention also relates to nucleic acid compounds for use in therapeutic and prophylactic methods in combination with an inhibitor of DNA synthesis. Such nucleic acid compounds may comprise or consist of a nucleic acid sequence capable of binding to a transferrin receptor (TfR).

The present invention provides nucleic acid compounds for use in a method of medical treatment or prophylaxis, wherein the method of medical treatment or prophylaxis comprises the step of administering the pharmaceutical composition in combination (i.e. simultaneously or sequentially) with an inhibitor of DNA synthesis. The invention also provides the use of nucleic acid compounds in the manufacture of medicaments for treating or preventing a disease or disorder, wherein the medicament is administered simultaneously or sequentially with an inhibitor of DNA synthesis. The invention described herein also provides methods of treating or preventing a disease or disorder, comprising administering to a subject in need thereof a therapeutically or prophylactically effective amount of a nucleic acid compound and an effective amount of an inhibitor of DNA synthesis.

Where components of a combination, e.g. a nucleic acid compound and an inhibitor of DNA synthesis, are administered together administration may be simultaneous administration. Where components of a combination, e.g. a nucleic acid compound and an inhibitor of DNA synthesis, are administered separately, administration may be simultaneous administration or sequential administration.

In some embodiments, the pharmaceutical compound comprising a nucleic acid compound which comprises or consists of a nucleic acid sequence capable of binding to a transferrin receptor (TfR) with a high affinity for TfR. In some embodiments, the nucleic acid sequence has an equilibrium dissociation constant (K^{D}) for TfR of less than about 1×10⁻⁸, 1×10⁻⁹, 1×10⁻¹⁰, 1×10⁻¹¹, or less than 1×10⁻¹² M. In some embodiments, the nucleic acid sequence has K^{D} for TfR of between 1×10⁻³ and 1×10⁻¹³ M, between 1×10⁻⁹ and 1×10⁻¹³ M, between 1×10⁻¹⁰ and 1×10⁻¹³ M, between 1×10⁻¹¹ and 1×10⁻¹³ M, or between 1×10⁻¹² and 1×10⁻¹³ M. In some embodiments, the nucleic acid sequence has a K^{D} for TfR of between 1 and 5×10⁻¹⁰ M, of between 1 and 5×10⁻¹¹ M, or of between 5×10⁻¹³ and 1×10⁻¹² M. In some embodiments, the nucleic acid sequence has a K^{D} for TfR of between 1×10⁻⁹ and 1×10⁻¹⁰ M, 1×10⁻¹⁰ and 1×10⁻¹¹ M, or of 1×10⁻¹² and 1×10⁻¹³ M.

In some embodiments, the nucleic acid compound finding use in combination with an inhibitor of DNA synthesis in the methods above comprises or consists of a nucleic acid sequence having at least 85% sequence identity to any one of SEQ ID NOs: 1 to 6. In some embodiments, the nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to any one of SEQ ID NOs:1 to 6. In some embodiments, the nucleic acid sequence has 85%, 86%, 87%, 88%, 89%, 90%, 91%, 92%, 93%, 94%, 95%, 96%, 97%, 98%, 99% or 100% sequence identity to any one of SEQ ID NOs: 1 to 6. In some embodiments, the nucleic acid sequence consists of any one of SEQ ID NO: 1, 2, 3, 4, 5 or 6. In some embodiments, the nucleic acid sequence comprises or consists of SEQ ID NO:12.

In some embodiments, the nucleic acid compound finding use in combination with an inhibitor of DNA synthesis comprises or consists of a nucleic acid sequence which hybridises with any one of SEQ ID NOs: 1 to 6. In some embodiments, the nucleic acid sequence hybridises with a sequence which has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to any one of SEQ ID NOs:1 to 6. In some embodiments, the nucleic acid sequence has 85%, 86%, 87%, 88%, 89%, 90%, 91%, 92%, 93%, 94%, 95%, 96%, 97%, 98%, 99% or 100% sequence identity to any one of SEQ ID NOs: 1 to 6. In some embodiments, the nucleic acid sequence consists of any one of SEQ ID NOs: 1 to 6. In some embodiments, the nucleic acid sequence comprises or consists of SEQ ID 7.

In some embodiments, the nucleic acid compound finding use in combination with an inhibitor of DNA synthesis comprises or consists of a nucleic acid sequence which is 87 nucleotides in length. In some embodiments, the nucleic acid sequence has a length of 46 nucleotides or fewer. In some embodiments, the nucleic acid sequence has a length of 43 nucleotides or fewer. In some embodiments, the nucleic acid sequence has a length of 40 nucleotides or fewer. In some embodiments, the nucleic acid sequence has a length of 32 nucleotides or fewer. In some embodiments, the nucleic acid sequence has a length of 22 nucleotides or fewer. In some embodiments, the nucleic acid sequence has a length of 16 nucleotides or fewer. In some embodiments, the nucleic acid sequence is 46 nucleotides in length. In some embodiments, the nucleic acid sequence is 43 nucleotides in length. In some embodiments, the nucleic acid sequence is 40 nucleotides in length. In some embodiments, the nucleic acid sequence is 32 nucleotides in length. In some embodiments, the nucleic acid sequence is 22 nucleotides in length. In some embodiments, the nucleic acid sequence is 16 nucleotides in length.

In some embodiments, the nucleic acid sequence is from 15 to 87, from 15 to 50, from 15 to 46, from 15 to 43, from 15 to 35, from 15 to 30, from 15 to 25, from 15 to 22, from 15 to 20, from 16 to 20, from 20 to 87, from 20 to 50, from 20 to 46, from 20 to 43, from 20 to 35, from 20 to 30, from 20 to 25, from 20 to 22, from 22 to 87, from 22 to 46, from 22 to 43, from 22 to 32, from 30 to 50, from 30 to 46, from 30 to 43, from 30 to 35, from 32 to 87, from 32 to 50, from 32 to 46, from 32 to 43, from 40 to 87, from 40 to 50, from 40 to 46, from 40 to 45, from 40 to 43, from 43 to 87, from 43 to 50, from 43 to 46, from 46 to 87, or from 46 to 50 nucleotides in length.

In some embodiments, the nucleic acid compound finding use in combination with an inhibitor of DNA synthesis comprises or consists of a nucleic acid sequence which has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer. In some cases the nucleic acid sequence has a length of 22 nucleotides or fewer. In some embodiments the nucleic acid sequence is between 16 and 29 nucleotides in length. In some embodiments the nucleic acid sequence is between 16 and 22 nucleotides in length.

In some embodiments, the nucleic acid compound finding use in combination with an inhibitor of DNA synthesis comprises or consists of a nucleic acid sequence which is 16 nucleotides in length. In some embodiments the nucleic acid sequence is 17 nucleotides in length. In some embodiments the nucleic acid sequence is 18 nucleotides in length. In some embodiments the nucleic acid sequence is 19 nucleotides in length. In some embodiments the nucleic acid sequence is 20 nucleotides in length. In some embodiments the nucleic acid sequence is 21 nucleotides in length. In some embodiments the nucleic acid sequence is 22 nucleotides in length. In some embodiments the nucleic acid sequence is 23 nucleotides in length. In some embodiments the nucleic acid sequence is 24 nucleotides in length. In some embodiments the nucleic acid sequence is 25 nucleotides in length. In some embodiments the nucleic acid sequence is 26 nucleotides in length. In some embodiments the nucleic acid sequence is 27 nucleotides in length. In some embodiments the nucleic acid sequence is 28 nucleotides in length. In some embodiments the nucleic acid sequence is 29 nucleotides in length.

In some embodiments, the nucleic acid compound finding use in combination with an inhibitor of DNA synthesis comprises or consists of a nucleic acid sequence which has at least 80% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 85% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 87% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 85% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 91% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 92% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 93% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 94% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 95% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 96% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 97% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 98% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 99% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer.

In some embodiments, the nucleic acid compound finding use in combination with an inhibitor of DNA synthesis comprises or consists of a nucleic acid sequence which has at least 100% sequence identity to SEQ ID NO:1 and has a length of 29 nucleotides or fewer, 28 nucleotides or fewer, 27 nucleotides or fewer, 26 nucleotides or fewer, 25 nucleotides or fewer, 24 nucleotides or fewer, 23 nucleotides or fewer, 22 nucleotides or fewer, 21 nucleotides or fewer, 20 nucleotides or fewer, 19 nucleotides or fewer, 18 nucleotides or fewer, 17 nucleotides or fewer, or 16 nucleotides or fewer. In some embodiments, the pharmaceutical composition comprises a nucleic acid compound according to any of the aspects herein.

In some embodiments, the nucleic acid compound finding use in combination with an inhibitor of DNA synthesis comprises or consists of a nucleic acid sequence having at least 85% sequence identity to any one of SEQ ID NOs 14 to 16. In some embodiments, the nucleic acid sequence comprises or consists of any one of SEQ ID NOs 14 to 16.

The nucleic acid compounds finding use in combination with an inhibitor of DNA synthesis may be conjugated to a moiety, as for any other nucleic acid compound as described herein. The nucleic acid compounds may be provided as pharmaceutical compounds may be formulated with one or more diluents, adjuvatives, carriers or stabilisers, as for any other pharmaceutical compound or formulation described herein.

In some embodiments, the nucleic acid compound finding use in combination with an inhibitor of DNA synthesis comprises a nucleic acid compound which is capable of being internalised into a cell.

The nucleic acid compound finding use in combination with an inhibitor of DNA synthesis may comprise a moiety attached to said nucleic acid sequence. The moiety may be attached to the 5′ or the 3′ end. In some embodiments, the moiety is attached to the 3′ end of the nucleic acid sequence. Any suitable moiety described herein may be used. In particular, the moiety is a therapeutic moiety, such as an anticancer therapeutic moiety. The therapeutic moiety is preferably covalently attached to the nucleic acid sequence. Suitable therapeutic moieties include a nucleic acid moiety, a peptide moiety or a small molecule drug moiety. For example, a therapeutic moiety may be a miRNA, mRNA, saRNA or siRNA moiety. Said therapeutic moiety may be a C/EBPalpha saRNA moiety, a SIRT1 saRNA moiety, or a HNF saRNA moiety. In particular, said therapeutic moiety may be a C/EBPalpha saRNA moiety.

In some embodiments, the nucleic acid compounds described herein find use in combination with an inhibitor of DNA synthesis in the treatment or prevention of cancer, metabolic disorders, or neurological disorders. In particular, the nucleic acid compounds and pharmaceutical compositions described herein may find use in combination with an inhibitor of DNA synthesis to treat or prevent cancer. The cancer may be any cancer as described herein (e.g. liver cancer (e.g. hepatocellular carcinoma), pancreatic cancer, pancreatic liver metastases, prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma). For example certain methods herein treat cancer by decreasing or reducing or preventing the occurrence, growth, metastasis, or progression of cancer; or treat cancer by decreasing a symptom of cancer. Symptoms of cancer (e.g. liver cancer (e.g. hepatocellular carcinoma), pancreatic cancer, pancreatic liver metastases, prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma) would be known or may be determined by a person of ordinary skill in the art.

In some embodiments, the cancer may be a pancreatic cancer, pancreatic liver metastases, pancreatic ductal adenocarcinoma (PDAC), acinar adenocarcinoma, intraductal papillary mucinous neoplasm (IPMN), pancreatic neuroendocrine tumors (PNETs), islet cell tumors, insulinoma, glucagonoma, gastrinoma, somatostatinoma, VIPomas, PPomas or pancreatoblastoma.

Compounds of the invention may find particular utility in combination with an inhibitor of DNA synthesis. This should be taken to encompass all drugs, prodrugs, conjugates, and derivatives thereof.

Exemplary inhibitors of DNA synthesis are described in Cozzarelli, Annual Review of Biochemistry, Volume 46, 1977, pp 641-668, which is herein incorporated by reference in its entirety. An inhibitor of DNA synthesis may be a DNA polymerase inhibitor or a ribonucleotide reductase inhibitor. An inhibitor of DNA synthesis may be a nucleoside analogue, e.g. a purine or pyrimidine nucleoside analogue. Exemplary compounds include 6-mercaptopurine, 5-fluorouracil (5-FU), capecitabine, tegafur, acycloguanosine (Acyclovir), acycloguanosyl 5′-thymidyltriphosphate, 2′-C-cyano-2′-deoxy-1-β-D-arabino-pentofuranosylcytosine (CNDAC), sapacitabine (a prodrug of CNDAC), doxifluridine, floxuridine, azacitidine (5-aza-2′-deoxycytidine), decitabine, or gemcitabine.

In particular embodiments, the inhibitor of DNA synthesis is gemcitabine (2′, 2′-difluoro 2′deoxycytidine) or a derivative or prodrug thereof. EP-A-0 184 365 (which is incorporated by reference in its entirety) discloses the synthesis of a range of novel 2′-deoxy, 2′,2′-difluoro nucleoside derivatives useful in the treatment of cancer, among these Gemcitabine is found. Derivatives of gemcitabine include gemcitabine esters or amides in which the 3′- and/or 5′-OH group and/or the N<4>-amino group is derivatised with a C18- and/or C20-saturated or mono-unsaturated acyl group, preferably an acyl group selected from oleoyl, elaidoyl, cis-eicosenoyl and trans-eicosenoyl, for example elaidic acid (5′)-gemcitabine ester and elaidic acid (N4)-gemcitabine amide, as detailed in EP0986570B1 (which is incorporated by reference in its entirety).

In particular embodiments, a nucleic acid compound as described herein and comprising a C/EBPalpha saRNA moiety finds use in combination with an inhibitor of DNA synthesis (e.g. gemcitabine) in the treatment or prevention of cancer. In some embodiments, the cancer is a pancreatic cancer, preferably PDAC.

### Sequence Identity

The terms “identical” or percent “identity,” in the context of two or more nucleic acid or polypeptide sequences, refer to two or more sequences or subsequences that are the same or have a specified percentage of amino acid residues or nucleotides that are the same (i.e., about 60% identity, preferably 65%, 70%, 75%, 80%, 85%, 87%, 90%, 91%, 92%, 93%, 94%, 95%, 96%, 97%, 98%, 99%, or higher identity over a specified region, when compared and aligned for maximum correspondence over a comparison window or designated region) as measured using e.g. a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from about 10 to 600, usually about 50 to about 200, more usually about 100 to about 150 in which a sequence may be compared to a reference sequence of the same number of contiguous positions after the two sequences are optimally aligned.

Methods of alignment of sequences for comparison are well known in the art. Optimal alignment of sequences for comparison can be conducted in various ways known to a person of skill in the art, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, PASTA, and FASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Publicly available computer software may be used such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

### Compound Moieties and Compounds

Nucleic acid compounds, e.g. ribo/deoxyribonucleic acid compounds, provided herein may comprise a therapeutic or diagnostic molecule.

The therapeutic or diagnostic molecule may form part of the nucleic acid compound provided herein, and is thus referred to as a “compound moiety”, e.g. a therapeutic moiety or an imaging moiety. Alternatively, the therapeutic or diagnostic molecule may not form part of the nucleic acid compound provided herein, including embodiments thereof, but may be independently internalised by a TfR-expressing cell upon binding of a nucleic acid compound provided herein to TfR on said cell. In this situation, the therapeutic or diagnostic molecule is referred to as a “compound.”

Thus, a nucleic acid compound provided herein (including embodiments thereof) may include a compound moiety. Where the nucleic acid compound includes a compound moiety, the compound moiety may be covalently (e.g. directly or through a covalently bonded intermediary) attached to the nucleic acid compound or the nucleic acid sequence (see, e.g., useful reactive moieties or functional groups used for conjugate chemistries set forth above). Thus, in some embodiments, the nucleic acid compound further includes a compound moiety covalently attached to the nucleic acid compound or the nucleic acid sequence. In embodiments, the compound moiety and the nucleic acid compound or the nucleic acid sequence form a conjugate. In some embodiments, the compound moiety is non-covalently attached to the nucleic acid compound or the nucleic acid sequence, e.g. via ionic bond(s), van der Waal's bond(s)/interactions, hydrogen bond(s), polar bond(s), “sticky bridges” (see e.g. Zhou J et al. Nucleic Acids Res. 2009; 37(9): 3094-3109) or combinations or mixtures thereof. The compound moiety may be attached to the nucleic acid compound or the nucleic acid sequence via an intermediate molecule such as a modular streptavidin connector (see e.g. Chu T C et al., Nucleic Acids Res 2006, 34:e73). Where the compound moiety is encapsulated as described herein, e.g. in a nanoparticle or liposome, the encapsulation moiety may itself be attached, covalently or non-covalently, to the nucleic acid compound or nucleic acid sequence.

In some embodiments, the compound moiety is a therapeutic moiety or an imaging moiety covalently attached to the nucleic acid compound or nucleic acid sequence.

The term “therapeutic moiety” as provided herein is used in accordance with its plain ordinary meaning and refers to a monovalent compound having a therapeutic benefit (prevention, eradication, amelioration of the underlying disorder being treated) when given to a subject in need thereof. Therapeutic moieties as provided herein may include, without limitation, peptides, proteins, nucleic acids, nucleic acid analogues, small molecules, antibodies, enzymes, prodrugs, nanostructures, viral capsids, cytotoxic agents (e.g. toxins) including, but not limited to ricin, doxorubicin, daunorubicin, taxol, ethidium bromide, mitomycin, etoposide, teniposide, vincristine, vinblastine, colchicine, dihydroxyanthracenedione, actinomycin D, diphtheria toxin, Pseudomonas exotoxin (PE) A, PE40, abrin, and glucocorticoid. In embodiments, the therapeutic moiety is an anti-cancer agent or chemotherapeutic agent as described herein. In embodiments, the therapeutic moiety is a nucleic acid moiety, a peptide moiety or a small molecule drug moiety. In embodiments, the therapeutic moiety is a nucleic acid moiety. In embodiments, the therapeutic moiety is a peptide moiety. In embodiments, the therapeutic moiety is a small molecule drug moiety. In embodiments, the therapeutic moiety is a nuclease. In embodiments, the therapeutic moiety is an immunostimulator. In embodiments, the therapeutic moiety is a toxin. In embodiments, the therapeutic moiety is a nuclease. In embodiments, the therapeutic moiety is a zinc finger nuclease. In embodiments, the therapeutic moiety is a transcription activator-like effector nuclease. In embodiments, the therapeutic moiety is Cas9. The therapeutic moiety may be encapsulated in a nanoparticle or liposome, where the nanoparticle or liposome is attached to the nucleic acid compound or the nucleic acid sequence.

In some embodiments, the therapeutic moiety is an activating nucleic acid moiety (a monovalent compound including an activating nucleic acid) or an antisense nucleic acid moiety (a monovalent compound including an antisense nucleic acid). An activating nucleic acid refers to a nucleic acid capable of detectably increasing the expression or activity of a given gene or protein. The activating nucleic acid can increase expression or activity 10%, 20%, 30%, 40%, 50%, 60%, 70%, 80%, 90% or more in comparison to a control in the absence of the activating nucleic acid. In certain instances, expression or activity is 1.5-fold, 2-fold, 3-fold, 4-fold, 5-fold,

10-fold or higher than the expression or activity in the absence of the activating nucleic acid. An antisense nucleic acid refers to a nucleic acid that is complementary to at least a portion of a specific target nucleic acid and is capable of reducing transcription of the target nucleic acid or reducing the translation of the target nucleic acid or altering transcript splicing. An antisense nucleic acid may be capable of detectably decreasing the expression or activity of a given gene or protein. The antisense nucleic acid can decrease expression or activity 10%, 20%, 30%, 40%, 50%, 60%, 70%, 80%, 90% or more in comparison to a control in the absence of the antisense nucleic acid.

In some embodiments, the therapeutic moiety is an miRNA moiety (a monovalent compound including a miRNA), an mRNA moiety (a monovalent compound including an mRNA), an siRNA moiety (a monovalent compound including an siRNA) or an saRNA moiety (a monovalent compound including an saRNA). In some embodiments, the therapeutic moiety is a miRNA moiety. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. In some embodiments, the therapeutic moiety is a siRNA moiety as described herein. In some embodiments, the therapeutic moiety is a saRNA moiety as described herein. In embodiments, the therapeutic moiety is an anticancer agent moiety. In some embodiments, the therapeutic moiety is an mRNA moiety. In embodiments, the therapeutic moiety is a cDNA moiety.

In some cases, the nucleic acid compound or the nucleic acid sequence provided herein is attached to a sense strand of a nucleotide compound moiety e.g., mRNA, miRNA, siRNA or saRNA. In some cases the nucleic acid compound or the nucleic acid sequence is attached to an antisense strand of a nucleotide compound moiety. In some cases, the nucleic acid compound or the nucleic acid sequence is attached to a guide strand of a nucleotide compound moiety. In some cases, the nucleic acid compound or the nucleic acid sequence is attached to a passenger strand of a nucleotide compound moiety. The moiety may be attached to the 5′ or the 3′ end. In some embodiments, the moiety is attached to the 3′ end of the nucleic acid sequence.

In some embodiments, the therapeutic moiety is a C/EBPalpha saRNA moiety. A “C/EBPalpha saRNA” as provided herein is a saRNA capable of activating and/or increasing the expression of a C/EBPalpha gene and/or C/EBPalpha protein. In some cases, for example, the saRNA sequence comprises SEQ ID NO:9 and/or SEQ ID NO:10.

In some embodiments, the therapeutic moiety is a sirtuin saRNA moiety. A “sirtuin saRNA” as provided herein is a saRNA capable of activating and/or increasing the expression of a sirtuin gene, e.g. SIRT1, SIRT2, SIRT3, SIRT4, SIRT5, SIRT6 or SIRT7. In some embodiments, the therapeutic moiety is a SIRT1 saRNA moiety. A “SIRT1 saRNA” as provided herein is a saRNA capable of activating and/or increasing the expression of a SIRT1 gene and/or a Sirt1 protein. In some cases, for example, the saRNA sequence comprises SEQ ID NO:7 and/or SEQ ID NO:8.

In some embodiments, the therapeutic moiety is a HNF saRNA moiety. A “HNF saRNA” as provided herein is a saRNA capable of activating and/or increasing the expression of a HNF gene and/or protein, for example HNF4 (including isoforms and variants thereof). An HNF saRNA may be an HNF4 saRNA. That is, an HNF saRNA may be one that modulates the expression, e.g. activates and/or increases the expression, of a HNF4 gene and/or protein.

In some cases, the therapeutic moiety may be NFκB (nuclear factor kappa-light-chain-enhancer of activated B cells) mRNA, miRNA, siRNA or saRNA.

In some cases, the therapeutic moiety may be a coenzyme such as NAD⁺/NADH (nicotinamide adenine dinucleotide), see for example Ying W, Front Biosci. 2007 Jan. 1; 12:1863-88.

The compound moiety provided herein may be an imaging moiety. An “imaging moiety” as provided herein is a monovalent compound detectable by spectroscopic, photochemical, biochemical, immunochemical, chemical, or other physical means. In some embodiments, the imaging moiety is covalently attached to the nucleic acid compound or the nucleic acid sequence. Exemplary imaging moieties are without limitation ³²P, radionuclides, positron-emitting isotopes, fluorescent dyes, fluorophores, antibodies, bioluminescent molecules, chemiluminescent molecules, photoactive molecules, metals, electron-dense reagents, enzymes (e.g., as commonly used in an ELISA), magnetic contrast agents, quantum dots, nanoparticles e.g. gold nanoparticles, biotin, digoxigenin, haptens and proteins or other entities which can be made detectable, e.g., by incorporating a radiolabel into a peptide or antibody specifically reactive with a target peptide. Any method known in the art for conjugating an antibody to the moiety may be employed, e.g., using methods described in Hermanson, Bioconjugate Techniques 1996, Academic Press, Inc., San Diego. Exemplary fluorophores include fluorescein, rhodamine, GFP, coumarin, FITC, Alexa fluor®, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese. In some embodiments, the imaging moiety is a bioluminescent molecule. In some embodiments, the imaging moiety is a photoactive molecule. In some embodiments, the imaging moiety is a metal. In some embodiments, the imaging moiety is a nanoparticle.

The term “imaging agent” as used herein describes the imaging moieties above when they are not attached to the nucleic acid compounds described herein.

In some cases, the nucleic acid compounds described herein comprise (i) an nucleic acid sequence as described herein and (ii) an additional aptamer molecule. Where said nucleic acid sequence is an aptamer, such molecules may be described as bispecific aptamers. Preferably, the additional aptamer molecule does not target and/or bind to TfR. In some cases, the nucleic acid compounds described herein are multivalent. In some cases, a terminus of a nucleic acid as described herein may be annealed to a terminus of an additional aptamer molecule using a complementary nucleotide linker sequence attached to each moiety (see e.g. McNamara, J. O. et al. J. Clin. Invest. 2008 118:376-386, which is hereby incorporated by reference in its entirety).

The compound moieties or compounds described herein may be conjugated to the nucleic acid compounds of the present invention by any suitable method as described herein or known in the art, see e.g. Zhu G et al., Bioconjug Chem. 2015 26(11): 2186-2197, hereby incorporated by reference in its entirety. Chemical-based linkers may employ activating reagent such as m-maleimidobenzoyl N-hydroxysuccinimide ester (MBS), 2-iminothiolane (Traut's reagent), N-succinimidyl-3-2-pyridyldithio propionate (SPDP) or may use e.g. PEGylation or avidin/biotin techniques (see e.g. Pardridge W M, Adv Drug Delivery Rev. 1999, 36:299-321; Qian Z M et al., which is hereby incorporated by reference in its entirety).

### Modifications

The nucleic acid compounds described herein may contain chemical modifications, e.g. as defined herein, to enhance their functional characteristics, such as nuclease resistance or binding affinity. The modifications may be present in a nucleic acid compound, a nucleic acid sequence and/or in a nucleotide-based compound moiety or compound, e.g. a saRNA, siRNA, miRNA, mRNA.

In some cases, modifications may be made to the base, sugar ring, or phosphate group of one or more nucleotides.

In some cases, the nucleic acid compounds described herein comprise one or more modified nucleobases. For example, the nucleic acid compounds may comprise one or more ribo/deoxyribo nucleobases modified with a fluoro (F), amino (NH₂) or O-methyl (OCH₃) group. In some cases, the nucleobases are modified at the 2′ position, the 3′ position, the 5′ position or the 6′ position. In some cases, the nucleic acid compounds may comprise one or more 2′-aminopyrimidines, 2′-fluoropyrimidines, 2′-O-methyl nucleotides and/or ‘locked’ nucleotides (LNA) (see e.g. Lin, Y et al., Nucleic Acids Res. 1994 22, 5229-5234 (1994); Ruckman, J. et al. J. Biol. Chem. 1998 273, 20556-20567; Burmeister, P E et al., Chem. Biol. 2005 12, 25-33; Kuwahara, M. & Obika, S. Artif. DNA PNA XNA 2013 4, 39-48; Veedu, R. N. & Wengel, J. Mol. Biosyst. 2009 5,787-792). In some cases, the nucleic acid compounds comprise one or more L-form nucleic acids (see e.g. Maasch, C et al., Nucleic Acids Symp. Ser. (Oxf.) 2008 52, 61-62). Other suitable nucleic acid modifications will be apparent to those skilled in the art (see, e.g. Ni S et al., Int. J. Mol. Sci 2017 18, 1683, hereby incorporated by reference in its entirety).

In some cases, a sense and/or antisense strand of a nucleotide compound moiety, e.g., mRNA, miRNA, siRNA or saRNA, may comprise a nucleotide overhang. For example, said overhang may be a 2-nucleotide (UU) overhang. Said overhang may be on the 3′ end of one or both strands. An overhang may favour Dicer recognition of the nucleotide compound moiety.

In some cases, the nucleic acid compounds described herein comprise an inverted thymidine cap on the 3′ end, or comprise 3′-biotin. In some cases, the phosphodiester linkage in the nucleic acid compounds in replaced with methylphosphonate or phosphorothioate analogue, or triazole linkages (see Ni S et al., supra).

In some cases, the nucleic acid compounds described herein comprise one or more copies of the C3 spacer phosphoramite. Spacers may be incorporated internally, e.g. between an nucleic acid sequence and a compound moiety, or at the 5′ or 3′ end of the nucleotide sequence to attach e.g. imaging moieties.

In some cases, the nucleic acid compounds described herein comprise modifications to increase half-life and/or resist renal clearance. For example, the compounds may be modified to include cholesterol, dialkyl lipids, proteins, liposomes, organic or inorganic nanomaterials, nanoparticles, inert antibodies or polyethylene glycol (PEG) e.g. 20 kDa PEG, 40 kDa PEG. Such modifications may be at the 5′-end of the compounds. In some cases, the modification comprises a molecule with a mass above the cut-off threshold for the renal glomerulus (˜30-50 kDa). In some cases, the nucleic compounds may be formulated with pluronic gel. For examples of suitable modifications and formulations see e.g. Ni et al, supra, and Zhou and Rossi, Nat Rev Drug Disc 2017, 16 181-202; both hereby incorporated by reference in their entirety.

The nucleic acid compounds described herein may comprise a tag, such as an albumin tag. An albumin tag may be attached to the nucleic acid compound at the nucleic acid sequence or at a moiety. A tag, such as an albumin tag, may be attached via a linker sequence, for example a poly-uridine (poly-U) linker. A poly-U linker may be about 20, about 19, about 18, about 17, about 16, about 15, about 14, about 13, about 12, about 11, about 10, about 9, about 8, about 7, about 6 or about 5 residues in length. A poly-U linker may be between 1 and 20, between 5 and 15, or between 8 and 12 residues in length.

Other tags may include: poly(His) tag, chitin binding protein (CBP), maltose binding protein (MBP), Strep-tag and glutathione-S-transferase (GST). The compounds may comprise an nucleic acid affinity tag, as described in, for example, Srisawat C and Engelke D R, Methods. 2002 26(2): 156-161 and Walker et al., Methods Mol Biol. 2008; 488: 23-40, hereby incorporated by reference in their entirety. Other suitable tags will be readily apparent to one skilled in the art.

The nucleic acid compounds described herein may comprise spacer or linker sequences between the nucleic acid portion and a compound moiety and/or tag. Suitable spacer or linker sequences will be readily apparent to one skilled in the art.

### Functional Characteristics

The nucleic acid compounds described herein may be characterised by reference to certain functional properties.

In some embodiments, any nucleic/ribonucleic/deoxyribonucleic acid compound described herein may possess one or more of the following properties:

Binds to transferrin receptor (TfR);

Capable of binding to TfR;

Binds specifically to TfR;

Capable of binding specifically to TfR;

Binds to TfR on the surface of a cell;

Capable of binding to TfR on the surface of a cell;

Capable of being internalised by a cell;

Capable of delivering a payload, e.g. compound moiety or compound, into a cell;

Capable of traversing the blood-brain barrier;

Capable of being transported into the brain;

Capable of delivering a payload, e.g. compound moiety or compound, into the brain.

The binding of a nucleic acid compound to a transferrin receptor can be determined by, e.g., surface plasmon resonance technology, as illustrated herein and described in Drescher et al., Methods Mol Biol. 2009; 493: 323-343.

The ability of a nucleic acid compound to be internalised by a cell or the ability to traverse the BBB can be determined using an imaging moiety conjugated to the nucleic acid compound, such as a fluorescent dye, and detecting said imaging moiety by an appropriate means. Suitable imaging methods are described herein or are well known in the art. Other methods include detecting a therapeutic moiety in brain tissue e.g. using an antibody.

The ability of a nucleic acid compound to deliver a payload into a cell can be determined by detecting the payload itself, e.g. by detection of an imaging moiety or otherwise as will be known in the art, or by detecting an effect of the successful delivery of said payload, e.g. as described herein.

The term “internalised,” “internalising,” or “internalisation” as provided herein refers to a composition (e.g., a compound, a nucleic acid compound, a therapeutic agent, an imaging agent) being drawn into the cytoplasm of a cell (e.g. after being engulfed by a cell membrane).

### Pharmaceutical Formulations

The present invention provides pharmaceutical compositions comprising the nucleic acid compounds described herein.

The nucleic acid compounds described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some cases, the nucleic acid compound according to the present invention are formulated for injection or infusion, e.g. into a blood vessel or tumour.

Pharmaceutical compositions of the nucleic acid compounds provided herein may include compositions having a therapeutic moiety contained in a therapeutically or prophylactically effective amount, i.e., in an amount effective to achieve its intended purpose. The pharmaceutical compositions of the nucleic acid compounds provided herein may include compositions having imaging moieties contained in an effective amount, i.e., in an amount effective to achieve its intended purpose. The actual amount effective for a particular application will depend, inter alia, on the condition being treated, tested, detected, or diagnosed. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically or prophylactically effective amount of a therapeutic moiety provided herein is well within the capabilities of those skilled in the art, especially in light of the detailed disclosure herein. When administered in methods to diagnose or detect a disease, such compositions will contain an amount of an imaging moiety described herein effective to achieve the desired result, e.g., detecting the absence or presence of a target molecule, cell, or tumour in a subject. Determination of a detectable amount of an imaging moiety provided herein is well within the capabilities of those skilled in the art, especially in light of the detailed disclosure herein.

The dosage and frequency (single or multiple doses) administered to a mammal can vary depending upon a variety of factors, for example, whether the mammal suffers from another disease; the route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein including embodiments thereof. Adjustment and manipulation of established dosages (e.g., frequency and duration) are well within the ability of those skilled in the art.

For any composition (e.g., the nucleic acid compounds provided, as well as combinations of an anticancer agent and the nucleic acid compound provided) described herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals.

The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

In one aspect, provided herein is a pharmaceutical composition including a nucleic acid compound as described herein, including embodiments thereof, and a pharmaceutically acceptable excipient. In some embodiments, the nucleic acid includes a compound moiety covalently attached to the nucleic acid compound or the nucleic acid sequence. As described above, the compound moiety may be a therapeutic moiety or an imaging moiety covalently attached to the nucleic acid compound or the nucleic acid sequence.

In some aspects, the pharmaceutical composition includes a nucleic acid compound as provided herein, including embodiments thereof, and a therapeutic agent. In some embodiments, the nucleic acid compound comprises a compound moiety. In some embodiments, the nucleic acid compound and the therapeutic agent are not covalently attached. A therapeutic agent as provided herein refers to a composition (e.g. compound, drug, antagonist, inhibitor, modulator) having a therapeutic effect. In some embodiments, the therapeutic agent is an anticancer agent. In some embodiments, the pharmaceutical composition includes a pharmaceutically acceptable excipient.

In some aspects, there is provided a pharmaceutical composition comprising a nucleic acid compound as provided herein, including embodiments thereof, and a compound as described herein. That is, the composition comprises the nucleic acid compound and a compound, e.g. a therapeutic or diagnostic molecule, which does not form part of the nucleic acid compound itself. In some cases, the nucleic acid compound comprises a compound moiety. In some cases, the pharmaceutical composition additionally comprises a therapeutic agent.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. a nucleic acid compound used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavours, salt solutions (such as Ringer's solution), alcohols, oils, gelatins, carbohydrates such as lactose, amylose or starch, fatty acid esters, hydroxymethycellulose, polyvinyl pyrrolidine, and colours, and the like. Such preparations can be sterilized and, if desired, mixed with auxiliary agents such as lubricants, preservatives, stabilizers, wetting agents, emulsifiers, salts for influencing osmotic pressure, buffers, colouring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “composition” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical composition is optionally in unit dosage form. In such form the preparation is subdivided into unit doses containing appropriate quantities of the active component. The unit dosage form can be a packaged composition, the package containing discrete quantities of composition, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

### Methods of Delivery

As described above the nucleic acid compounds, e.g. ribo/deoxyribonucleic acid compounds provided herein, including embodiments thereof, may be used to deliver compound moieties or compounds (e.g., therapeutic agents or imaging agents) into a cell. Where a compound moiety (e.g., therapeutic moiety or imaging moiety) is delivered into a cell, the compound moiety may be covalently attached to the nucleic acid compound provided herein including embodiments thereof. Upon binding of the nucleic acid compound to TfR on a cell, the compound moiety may be internalized by the cell while being covalently attached to the nucleic acid compound. Thus, in one aspect, a method of delivering a compound moiety into a cell is provided. The method includes, (i) contacting a cell with the nucleic acid compound, or composition, as provided herein including embodiments thereof and (ii) allowing the nucleic acid compound to bind to a TfR on the cell and pass into the cell thereby delivering the compound moiety into the cell.

Alternatively, where a compound is delivered into a cell, the compound (e.g., a therapeutic agent or an imaging agent) may not be covalently attached to the nucleic acid compound. Upon binding of the nucleic acid compound provided herein, including embodiments thereof, to TfR on a cell, the nucleic acid compound and the compound provided may be internalized by the cell without being covalently attached to each other. Thus, in another aspect, a method of delivering a compound into a cell is provided. The method includes (i) contacting a cell with a compound and the nucleic acid compound, or composition, as provided herein including embodiments thereof and (ii) allowing the nucleic acid compound to bind to a TfR on the cell and the compound to pass into the cell thereby delivering the compound into the cell. In embodiments, the compound is a therapeutic agent or imaging agent. In embodiments, the compound is non-covalently attached to the nucleic acid compound.

The methods may be performed in vitro, ex vivo, or in vivo. In some cases, the methods comprise delivering the compound moiety or compound across the blood-brain barrier into the brain.

### Therapeutic and Prophylactic Applications

The nucleic acid compounds, e.g. ribo/deoxyribonucleic acid compounds, and compositions provided herein find use in therapeutic and prophylactic methods.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The nucleic acid compounds described herein find use in the treatment or prevention of any disease/disorder which would benefit from the delivery of said compounds, and/or associated therapeutic or imaging moieties, to cells expressing TfR. The nucleic acid compounds also find use in the treatment or prevention of any disease/disorder which would benefit from the delivery of said compounds and/or associated moieties to the brain.

It will be appreciated that the therapeutic and prophylactic utility of the present invention extends to the treatment of any subject that would benefit from the delivery of a compound moiety or compound into a cell expressing TfR, or into the brain.

In some embodiments, the disease/disorder is one which would benefit from the activation of a Sirtuin gene/protein e.g. SIRT1, the activation of a C/EBPalpha gene/protein, and/or the activation of a HNF gene/protein. In some embodiments, the nucleic acids and compositions described herein find use to treat or prevent cancer, metabolic disorders, or neurological disorders.

For example, in some embodiments, certain methods described herein treat cancer (e.g. liver cancer (e.g. hepatocellular carcinoma), pancreatic cancer, pancreatic liver metastases, prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma). For example certain methods herein treat cancer by decreasing or reducing or preventing the occurrence, growth, metastasis, or progression of cancer; or treat cancer by decreasing a symptom of cancer. Symptoms of cancer (e.g. liver cancer (e.g. hepatocellular carcinoma), pancreatic cancer, pancreatic liver metastases, prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or oesophagus), colorectal cancer, leukaemia, acute myeloid leukaemia, lymphoma, B cell lymphoma, or multiple myeloma) would be known or may be determined by a person of ordinary skill in the art.

In some embodiments, the cancer is a cancer as described herein. In some embodiments, the cancer is liver cancer e.g. hepatocellular carcinoma, pancreatic cancer, pancreatic liver metastases, metastatic cancer, or brain cancer.

In some cases, the cancer is one in which activation of a Sirtuin gene/protein e.g. SIRT1, activation of a C/EBPalpha gene/protein, and/or activation of a HNF gene/protein has a therapeutic or prophylactic effect.

In some embodiments, the methods of treatment described herein comprise administering to a subject in need thereof a therapeutically or prophylactically effective amount of a nucleic acid compound or composition as described herein, wherein the nucleic acid compound comprises an anticancer therapeutic moiety. In some embodiments, the methods of treatment further comprise administering to a subject in need thereof an effective amount of an anticancer agent.

In some cases, the methods of treatment described herein comprise inducing or inhibiting autophagy, for example through the activation or inhibition of Beclin1. See e.g. Jin and White, Autophagy 2007; 3(1):28-31; Rosenfeldt and Ryan, Expert Rev Mol Med. 2009; 11:e36; and Mah and Ryan, Cold Spring Harb Perspect Biol. 2012; 4(1): a008821, all hereby incorporated by reference in their entirety. In some cases, the methods of treatment described herein comprise inducing or inhibiting the activity of nuclear factor kappa-light-chain-enhancer of activated B cells (NF-κB).

In some embodiments, the disease/disorder is a metabolic disorder. For example, the metabolic disorder may be metabolic syndrome, type I diabetes mellitus, type 2 diabetes mellitus, dyslipidemia, impaired fasting glucose, impaired glucose tolerance, obesity, cardiovascular disease, insulin resistance, hypertriglyceridemia, psoriasis, psoriatic arthritis, coronary vascular diseases e.g. coronary heart disease, coronary artery disease, stroke and peripheral artery disease, atherosclerosis, fatty liver disease, non-alcoholic fatty liver disease (NAFLD), steatohepatitis, and/or lipodystrophic disorders.

In some cases, the metabolic disorder is one in which activation of a Sirtuin gene/protein, activation of a C/EBPalpha gene/protein, and/or activation of a HNF gene/protein has a therapeutic or prophylactic effect.

In some cases, the nucleic acids and compositions of the present invention find use in the reduction of body weight, reduction of body weight gain, reduction of serum glucose, regulating glucose homeostasis, decreasing insulin resistance, reduction of white adipose tissue, reduction of cholesterol, reduction of low-density lipoprotein (LDL), increasing high-density lipoprotein (HDL), increasing high-density lipoprotein/low-density lipoprotein (HDL/LDL) ratio, reduction of serum triglycerides.

In some cases, the nucleic acids and compositions of the present invention find use in targeting TfR-expressing cells in the pancreas, brain, heart, white and brown adipose tissue, muscle, and/or liver.

In some embodiments, the disease/disorder is a neurological disorder. For example, the neurological disorder may be Alzheimer's disease, amyotrophic lateral sclerosis (ALS), motor neuron disease, Parkinson's disease, Huntington's disease, spinal and bulbar muscular atrophy (SBMA).

In some cases, the neurological disorder is one in which activation of a Sirtuin gene/protein, activation of a C/EBPalpha gene/protein, and/or activation of a HNF gene/protein has a therapeutic or prophylactic effect.

In some cases, the nucleic acids and compositions of the present invention find use in the treatment or prevention of, i.e. reduction of or protection against, neurodegeneration.

Where combination treatments are contemplated, it is not intended that the agents (i.e. nucleic acid compounds) described herein be limited by the particular nature of the combination. For example, the agents described herein may be administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount”. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient”, “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, monkeys, goat, sheep, cows, deer, and other non-mammalian animals. In some embodiments, a patient is human.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The nucleic acid compounds described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

### Methods of Detecting a Cell

The nucleic acid compositions, e.g. ribo/deoxyribonucleic acid compounds, provided herein may also be used for the delivery of compounds and compound moieties to a cell expressing TfR. As described above, the compounds and compound moieties delivered may be imaging agents useful for cell detections. Thus, in one aspect, a method of detecting a cell is provided. The method includes (i) contacting a cell with the nucleic acid compound, or composition, as provided herein including embodiments thereof, wherein the nucleic acid compound further includes an imaging moiety, (ii) the nucleic acid compound, or composition, is allowed to bind to a transferrin receptor on the cell and pass into the cell, (iii) the imaging moiety is detected thereby detecting the cell.

In another aspect, a method of detecting a cell is provided. The method includes (i) contacting a cell with an imaging agent and the nucleic acid compound, or composition, as provided herein including embodiments thereof, (ii) the nucleic acid compound, or composition, is allowed to bind to a transferrin receptor on the cell and the imaging agent is allowed to pass into the cell, (iii) the imaging agent is detected thereby detecting the cell.

In some cases, the cell is a malignant cell. In some cases, the cell is a breast cancer cell. In some cases, the cell is a prostate cancer cell. In some cases, the cell is a liver cancer cell. In some cases, the cell is a pancreatic cancer cell. In some cases, the cell is a brain cancer cell. In some cases, the cell is a non-malignant cell. In some cases, the cell is a brain cell. In some cases, the cell forms part of an organism. In some cases, the organism is a mammal. In some cases, the cell forms part of a cell culture.

The methods may be performed in vitro, ex vivo, or in vivo.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Materials and Methods

Chemicals and reagents. Chlorpromazine (CPZ, #C8138), chloroquine (CQ, #C6658), dynasore (#D7693), genistein (GEZ, #D6649), cytochalasin D (Cyto D, #C8273), and HBSS (#6648) were purchased from Sigma. Cell light early endosome-GFP (C10588), late endosome-GFP (C10586), and lysosome-GFP (C10507) were purchased from ThermoFisher scientific. Anti-transferrin receptor antibodies (ab47095) were purchased from Abcam. Human transferrin-Alexa488 was purchased form Invitrogen (1780257).

Recombinant target protein. hTfR was purchased from Sino Biological Inc (11020-H07H, Beijing, P.R. China). The extracellular domain of hTfR (NP_003225.2) (Cys 89-Phe 760) was expressed with a His6-tag at the N-terminus in human cells (HEK293).

Cell line. HepG2 (Hepatocarcinoma, HB-8065) and PANC-1 (Pancreatic epithelioid carcinoma, CRL-1469), U-87 MG (Glioblastoma, HTB-14) were purchased from the American Type Culture Collection (ATCC, Manassas, Va.). The cells were cultured according to the suppliers' instructions. TB10 human glioma cell line was obtained from Vittorio de Franciscis lab in Italy. The cells were cultured according to the suppliers' instructions.

Protein SELEX. In vitro selection was carried out as described in Yoon et al, 2010, with a few modifications. The 2′F-RNA aptamers were selected from 40-nucleotide randomized sequences constructed by in vitro transcription of synthetic DNA templates with NTPs (2F UTP, 2′F CTP, GTP, ATP; Epicentre Biotechnologies, Madision, Wis.) and T7 RNA polymerase. To remove RNAs that bound nonspecifically to agarose beads, 1.44 μM of the RNA library was pre-incubated with 20 μl of Ni-NTA agarose beads in 100 μl binding buffer (30 mM Tris-HCl, pH 7.5; 150 mM NaCl; 5 mM MgCl2; 2 mM dithiothreitol; 1% BSA; 100 μg/mL yeast tRNA) for 30 min at room temperature with shaking, precipitated by centrifugation, and discarded. The precleared supernatant was transferred to a new tube and incubated with 300 nM of His6-tagged hTFRC for 30 min at room temperature. RNAs that bound to hTFRC were recovered, amplified by RT-PCR and in vitro transcription, and used in subsequent selection rounds. In subsequent rounds, hTFRC concentration was reduced by 2-fold at every three rounds for more stringent conditions. After nine rounds of SELEX, the resulting cDNA was amplified. The amplified DNA was cloned and individual clones were identified by DNA sequencing. Aptamer structures were predicted using Mfold (see Zuker et al 2003, available at http://www.bioinfo.rpi.edu/applications/mfold/ using a salt correction algorithm and temperature correction for 25° C., or were predicted using NUPACK (Zadeh et al, 2011, available at http://www.NUPACK.org).

Truncation of anti-TfR aptamers. To generate the TR14 truncations, the sequence of full-length TR14 was loaded into the program Mfold (Zuker, 2003), available at http://www.bioinfo.rpi.edu/applications/mfold/, using a salt correction algorithm and temperature correction for 25° C. Using a computer-guided approach, bases were removed from the 5′ and 3′ ends until the predicted secondary structure of the remaining oligonucleotide was as similar as possible to that of full-length TR14. To create the illustrations, the secondary structure was rendered with the program NUPACK (Zahed et al, 2011), available at http://www.NUPACK.org.

Surface plasmon resonance-based biosensor assay. The Biacore T100 (GE Healthcare, Uppsala, Sweden) was used to monitor label-free interactions of truncated TR14-hTfR1 in real time. The biotinylated aptamer was coupled to a streptavidin-coated Biacore chip (SensorChip SA, BR-1003-98, General Electric Company) by an injection in binding buffer at a concentration of 25 μg/mL (30 mM Tris-HCl, pH 7.5; 150 mM NaCl; 5 mM MgCl₂) at 10 uL/min. The RNA was refolded by heating to 65° C., followed by cooling to 37° C., before immobilization. To measure binding kinetics, five concentrations of purified hTfR1 protein were injected at a flow rate of 10 uL/min. After binding, the surface was regenerated by injecting 50 mM NaOH at a flow rate of 15 μL/min for 20 s. Data from the control surface were subtracted. BIAevaluation software (GE Healthcare) was used for analysis. The binding data was fit to a 1:1 binding with a mass transfer model to calculate kinetics parameters as previously described (Hernandez et al, 2009; Soontornworajit et al, 2011)

Live-cell confocal imaging for aptamer internalization. 1×10⁵ HepG2 or PANC-1 cells were seeded in 35-mm glass-bottom dishes (MatTek, Ashland, Mass.) and grown in appropriate media for 24 h. Aptamer RNAs were labeled with Cy3 fluorescent dye using the Cy3 Silencer siRNA labeling kit (Thermo Fisher Scientific, Waltham, Mass.). Cy3-labeled aptamers in binding buffer (30 mM Tris-HCl, pH 7.5; 150 mM NaCl; 5 mM MgCl₂; 100 μg/mL yeast tRNA) were added to the cells at 200 nM and incubated at 37° C. for 2 h. Before imaging, cells were washed with DPBS twice. Live-cell confocal imaging was performed with a Zeiss LSM 510 Meta inverted two-photon confocal microscope system using a C-Apo 40×/1.2NA water immersion objective, and AIM 4.2 software (Carl Zeiss, Jena, Germany).

Flow cytometry-based binding assays. Aptamer binding was assessed by flow cytometry. For the assay, the PANC-1 cells were detached using Accutase, washed with PBS and suspended in binding buffer. Next, chemically synthesized aptamers labeled with Cy3 at 500 nM were added to target cells for 20 minutes in ice. Cells were washed with binding buffer and immediately analyzed by NovoCyte (ACEA Biosciences). For the exclusion of dead cells, 4′6′-diamidino-2-phenylindole (DAPI) (1 μg/ml) was used. The data were analyzed with NovoExpress software.

Cellular uptake inhibition assay. 5×103 PANC-1 cells/well were seeded in 96-well plates one day before assay. PANC-1 cells were left either pretreated or untreated with chlorpromazine (CPZ, clathrin endocytosis inhibitor, 10 μg/mL), chloroquine (CQ, clathrin endocytosis inhibitor, 20 μg/mL), dynasore (dynamine inhibitor, 80 μM), genistein (GEZ, caveolae/lipid mediated endocytosis inhibitor, 50 μg/mL), or, cytochalasin D (Cyto D, 5 μM) at 37° C. for 30 min. Subsequently, cells were washed with DPBS and incubated with Cy3-labeled aptamers at 200 nM at 37° C. f for 1 hour. Following incubation, cells were washed with HBSS to remove surface-bound aptamers. Cellular uptake was quantified by fluorescence using a plate reader (Molecular device, SpectraMax iD3). The inhibition of uptake was normalized with inhibitor non-treated groups (mock)

Competition assays. For competition assays with human transferrin or human transferrin receptor antibodies, 5×103 PANC-1 cells/well were seeded in 96-well plates and grown in one day. Cells were pre-incubated with TR14 at 200 nM for 20 min on ice with PBS. After washing with PBS, human transferrin conjugated with Alexa-488 at 25 μg/mL or human transferrin receptor antibody (2 μL/1×104 cells) conjugated with FITC were added to cells and the cells were incubated at 37° C. for 40 min. Afterward, the cells were washed with HBSS twice to remove cell surface binders. The intensity of fluorescence was quantified using plate reader (Molecular device, SpectraMax iD5). The intensity was normalized with aptamer non-treated control cells (mock).

Co-localization assay. 1×105 PANC-1 cells were seeded in 35 mm glass-bottom dishes (MatTek, Ashland, Mass.) and grown in appropriate media for 24 h. CellLight™ Early endosome-GFP BacMam 2.0 (Thermo Fisher, C10586, CellLight™ Late endosome-GFP BacMam 2.0 (Thermo Fisher, C10588), or CellLight™ Lysosome-GFP BacMam 2.0 lysosome-GFP (Thermo Fisher, C10596) of 20 μL were added and incubated at 37° C. for 16 h. After confirmation of GFP expression, chemically synthesized TR14 labelled with Cy3 at 200 nM was incubated at 37° C. for 2 h. The co-localization was assessed on live cells by confocal microscopy.

Aptamer conjugation to C/EBPα-saRNA. The tTR14-C/EBPα-sense strand with or without albumin affinity tag, and antisense RNA of C/EBPα were chemically synthesized at City of Hope. The RNAs were refolded in binding buffer, heated to 95° C. for 3 min, slowly cooled to 37° C., then incubated at 37° C. for 10 min. To form the tTR14-C/EBPα RNAs, antisense strand of C/EBPα were annealed to the complementary strand using the same molar amounts. The same amount of refolded tTR14-was added and incubated at 37° C. for 10 min in binding buffer to make the chimeric conjugates.

A “sticky” sequence (a 16-nucleotide sequence that prevents structural hindrance) was placed between TR14 and the C/EBPα-saRNA oligonucleotide, as described in Yoon et al, 2016. TR14-STICK-sense, P19-STICK, control-STICK, Sense-STICK, and antisense RNAs were chemically synthesized. The TR14-STICK, P19-STICK, or control-STICK RNAs were refolded in binding buffer, heated to 95° C. for 3 min, slowly cooled to 37° C., then incubated at 37° C. for 10 min. To form the STICK-C/EBPα RNAs, the sense-STICK and antisense strand were annealed to the complementary strand using the same molar amounts. The same amount of refolded TR14-, P19-, or control-STICK was added and incubated at 37° C. for 10 min in binding buffer to make the chimeric conjugates. For truncated TR14, the same molar amounts of aptamer-STICK-sense and CEBPA anti-sense were annealed in binding buffer by heating to 95° C. for 3 min and slowly cooled to 37° C.

Chemical synthesis of albumin tag to aptamers. 4-(p-Iodophenyl) butyric acid from Sigma was converted into NHS ester using standard procedure. 4-(p-Iodophenyl) butyric acid NHS ester was used for the conjugation with 3′-amino or 5′-amino labeled oligonucleotides. 1.0 μmole of oligonucleotide was dissolved in 750 uL of water, 400 uL of DMSO was added, followed by 150 uL of 1.0 M phosphate buffer pH 7.5. Solution of 5 eq. of 4-(p-Iodophenyl) butyric acid NHS ester in 100 uL of DMSO was added. Reaction mixture was vigorously stirred at room temperature. Progress of the reaction was monitored by HPLC on the PRP1 column (Hamilton) in TEAA buffers. Buffer A: 50 mM TEAA in water, Buffer B: 50 mM TEAA in acetonitrile-water, 9:1. After 1 hour reaction was completed. In order to remove the DMSO reaction mixture was precipitated into isopropanol, kept at −20° C. overnight, centrifuged at 4° C. for 20 min. Supernatant was discarded and the resulted palette was washed with cold ethanol:water, 8:2.

Relative gene expression analysis by qPCR in vitro. For analyzing gene activation and protein expression, PANC-1 cells were seeded in duplicate into 24-well plates at a density of 1×10⁵ cells per well. tTR14 or control aptamer (IRRE) conjugated to C/EBPα-saRNAs were added directly to the cells, at a final concentration of 100 nM or 200 nM. The treatment was repeated 24 h later and cells were harvested at final incubation times of 48 or 72 h. Total RNA was extracted for reverse transcription using an RNeasy kit (Qiagen). Target cDNA amplification and real-time PCR was performed using a Bio-rad kit (SsoAdvanced™ Universal SYBR® Green Supermix). For normalization, the reference gene 18S was used. To determine gene expression in vivo samples, total RNA was extracted for reverse transcription (QuantiFast® Reverse transcription, Qiagen) and target cDNA was amplified using real-time PCR (QuantiFast® SYBR®Green Master mix). The cDNA probes used were purchased as prevalidated QuantiTect® SYBR probes from Qiagen. Real-time PCR was performed with validated QuantiTect® SYBR probes (Qiagen) or validated FAM probes (Applied Biosystems).

MTS assay. To determine the inhibition of cell proliferation, 5×10³ PANC-1 cells/well were seeded in 96-well plates and grown in appropriate media one day before the treatment. Cells were treated with tTR14-C/EBPα and IRRE-tTR14-C/EBPα at 100 nM or 200 nM twice at 24-h intervals. Inhibition of cell proliferation was measured using MTS assay (Promega, Madison, Wis.) at a final incubation time of 48 or 72 h.

Analysis of gene expression in vivo. Female or male at 6 week C57BL/6J mice were purchased from the Jackson laboratory for three conjugates; TC, TCT, and TCUT. For these assays, C57BL/6J mice were housing at animal facility at the City of Hope until sacrificing at the end of time point. To determine the expression of C/EBPα in tissues of interest, 1 nmol of each conjugates was injected via tail-vein injection every other day three times (D1, D3, and D5). After three days on last injection (D8), each tissue sample was collected and quick frozen for further analysis.

Intrahepatic pancreatic cancer liver-metastatic mouse model. To create an animal model harboring traceable tumors, a firefly luciferase fragment was inserted into the pLKO.1-AS3 backbone encoding the neo gene (National RNAi Core, Academia Sinica, Taiwan). One day before transduction, 293T cells were plated onto a 6-well plate. On the day of transduction, the medium was replaced with DMEM containing serial dilutions of the transfer plasmids and incubated for 5 h, then the medium was replaced. After two days, the culture medium containing recombinant lentiviral particles was obtained. PANC-1 cells were incubated with recombinant lentiviral particles for 24 h. The following day, culture medium was replaced with standard medium containing 1.2 mg/mL G418 (Merck, Germany) for stable clone selection. Two weeks after selection, a single stable cell line was picked and maintained in medium containing G418. Luciferase expression was assessed using the Luciferase Assay System.

Six-week-old female NOD/SCID mice (BioLasco Co., Taiwan) were used in these experiments. Animal studies were performed in compliance with approval from the Institutional Animal Care and Use Committee of College of Medicine, National Taiwan University. Mice were kept in a conventional, specific pathogen-free facility. To establish a liver-metastatic pancreatic cancer model, intrahepatic tumor implantation was performed by injecting 30 μL of a monocellular suspension (in PBS) containing 1×10⁶ PANC-Luc cells into a region in the middle lobe of the livers of 6-week-old female NOD/SCID mice (BioLasco Co., Taiwan). Tumors were allowed to grow for 1 week after inoculation, then the mice were randomly divided into five groups and injected with PBS, IRRE-TR14-CEBPA (1 nmol), TR14-CEBPA (1 nmol), IRRE-P19-CEBPA (1 nmol) or P19-CEBPA (1 nmol) via tail vein 3 times/week for 3 weeks.

For the combinational treatment with gemcitabine, tumors were allowed to grow for 2 weeks after inoculation until they could be detected using an IVIS system, then mice were randomly divided into five groups of 10 animals/group. Each treatment group followed a specific schedule. The Gem group was treated with gemcitabine (50 mg/kg, 2 times/week, I.V.). The Gem/TC group was treated with gemcitabine (50 mg/kg, 2 times/week, I.V.) and TC (1 nmol, 3 times/week, I.P.). The Gem/TCT group was treated with gemcitabine (50 mg/kg, 2 times/week, I.V.) and TCT (1 nmol, 3 times/week, I.P.). The Gem/TCUT group was treated with gemcitabine (50 mg/kg, 2 times/week, I.V.) and TCUT (1 nmol, 3 times/week, I.P.). The control group was treated with PBS only.

Tumor growth was monitored by evaluating bioluminescence using an IVIS 200 in vivo imaging platform (Caliper Life Sciences, Alameda, Calif.) and measuring the difference from before the first injection and one day after the last injection. To do this, prior to in vivo imaging, the mice were anesthetized using isoflurane. A solution of 150 μg/kg D-luciferin (Biosynth, USA) was then injected intraperitoneally. The mice were imaged using the IVIS 200 and bioluminescent signals were analyzed using Living Image Software (Caliper Life Sciences, Alameda, Calif.). The mice were euthanized two days after the last injection. Tumors were removed from mice and tumor size was measured by caliper and further analysis of gene expression.

Statistical analysis. Data were presented as means±S.D. Statistically significant differences were determined by two-tailed Student's t-test, ANOVA, or unpaired t-test with Welch's correction using Graph Pad Prism software (GraphPad Software, La Jolla, Calif., USA). The difference was considered significant when the p value was less than *: P≤50.05, **: P≤50.01.

### Example 1—Generation of Anti-hTFRC Aptamer TR14 Through Protein SELEX

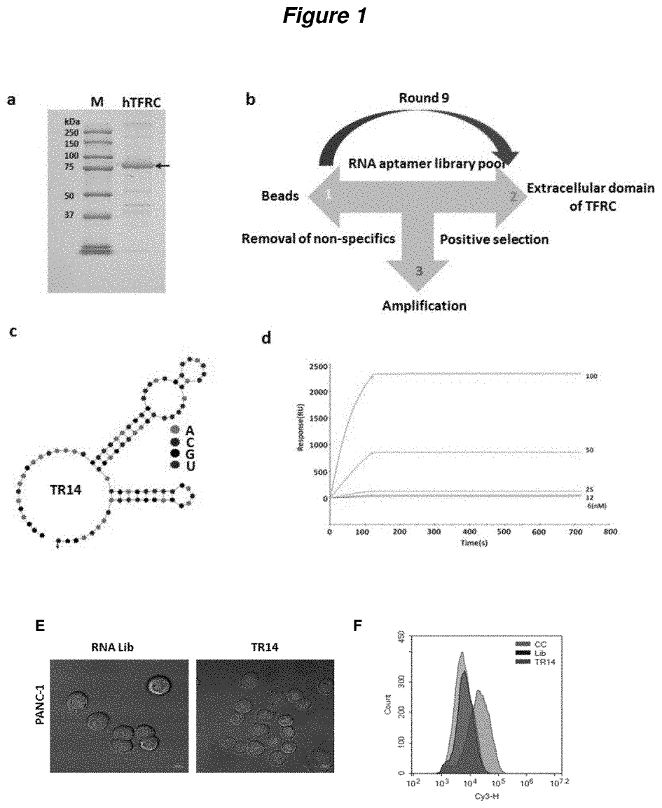

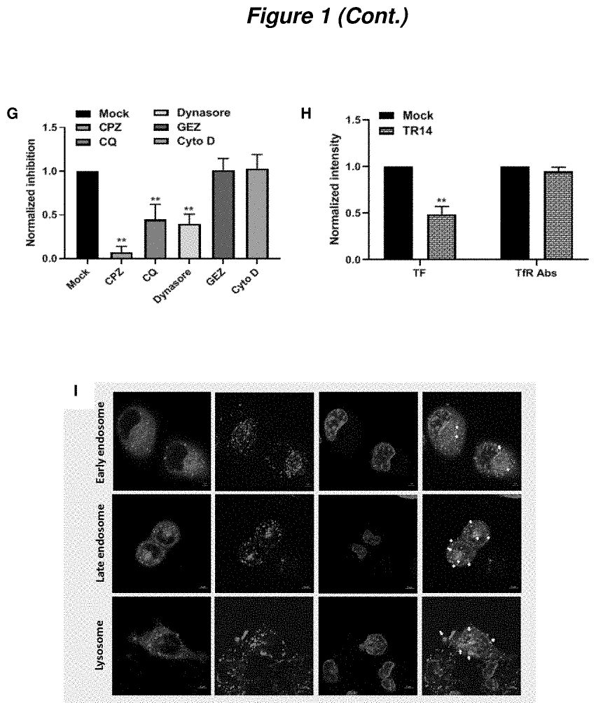

We used protein SELEX to select RNA aptamers against hTfR. As a target for SELEX, the extracellular domain of hTfR with a polyhistidine (His6) tag to immobilized to beads was expressed in human embryonic kidney (HEK293) cells. The SDS-PAGE and Coomassie stain was used to confirm that the expected size of the target protein was 75 kDa (FIG. 1A).

We first incubated an RNA aptamer library pool with agarose beads to remove non-specific binders. Subsequently, we incubated the supernatant with the His6-hTFRC target protein for positive selection, then amplified the aptamers bound to hTfR using PCR and in vitro transcription, as depicted in FIG. 1b.

After nine rounds of SELEX, we identified the 87-nucleotide anti-hTfR aptamer TR14, 5′-GGGAGACAAGAAUAAACGCUCAAUGCGUUCACGUUUAUUCACAUUUUUGAAUUGAGCAUGAGCU UCGACAGGAGGCUCACAACAGGC-3′. We predicted the structure of TR14 using Mfold, which showed multiple stem-loop structures (FIG. 1C). To characterize TR14-hTfR binding affinity and kinetics, we used a label-free biosensor assay in real-time.

We measured the equilibrium dissociation constant (K^{D}) of TR14 for hTfR as 3.17×10⁻¹¹ mol/L, with 6.78×10³ M⁻¹s⁻¹ and 1.68×10⁻⁶ s⁻¹ as the corresponding association (ka) and dissociation (kd) rate constants, respectively (FIG. 1D and Table 2).

### Example 2—Generation of Truncated TR14s

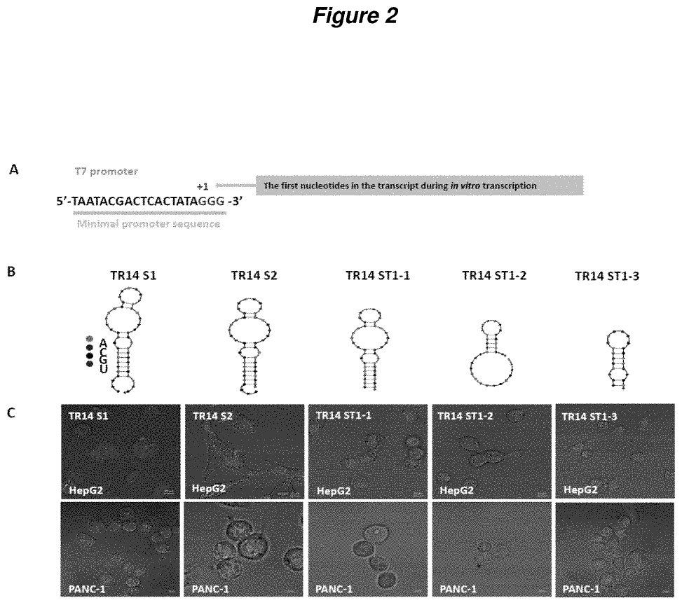

Based on structural analysis using computational prediction, we truncated TR14 into the smallest functional unit that was expected to maintain binding to hTfR. We generated five TR14 truncates: S1 (46-nt), S2 (43-nt), ST1-1 (40-nt), and ST1-2 (32-nt), ST1-3 (22-nt) (Table 1). During the SELEX process, in vitro transcription was used to generate RNA transcripts with the T7 RNA polymerase promoter region “TAATACGACTCACTATAGGGAGA”. As depicted in the procedure of synthesis of RNA by in vitro transcription (Yoon & Rossi, 2017) the T7 promoter covers the sequences from −17 with +1 being the first nucleotides of transcribed region (FIG. 2A). To generate the truncates of hTfR RNA aptamers, we put the minimum consensus sequences of T7 RNA polymerase promoter “TAATACGACTCACTATAGGG” in vitro transcription step. Hence, TR14 S1 and TR14 S2 sequences contained the first three G's at the 5′. To determine the effect of the first three G's transcribed from the T7 promoter region during in vitro transcription, TR14 ST1-1, TR14 ST1-2, and TR14 ST1-3 without 5′-GGG were chemically synthesized. The expected structures of the truncates were generated using NUPACK (FIG. 2B).

### Example 3—the Anti-hTFRC Aptamer TR14 and Truncated TR14s are Efficiently Internalized into Cancer Cells

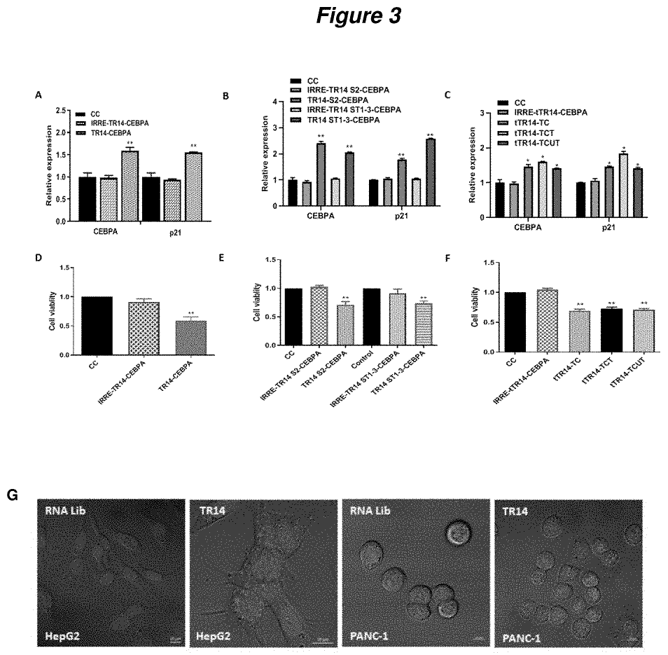

We performed cell internalization assays using confocal microscopy of live cells to confirm the efficacy of TR14 for targeted delivery. After labeling TR14 with Cy3, we treated the liver cancer cell line HepG2 and the pancreatic cancer cell line PANC-1 with TR14 (200 nM) that is overexpressed of hTFRC. For the control, Cy3 labeled initial RNA library was treated on both cell lines. After a 2-h incubation, we observed a typical punctate fluorescence pattern in the cytoplasm, suggesting that TR14 was successfully internalized into both types of cancer cells (FIGS. 1E and 3G).

To confirm the binding of TR14 to cancer cells, Cy3-labelled initial non-selected aptamer library as negative control or TR14 aptamers were incubated on PANC-1 cells. We observed enriched cell surface binding to the cells, compared with initial RNA library pool by flow cytometry (FIG. 1F).

To characterize TR14-hTfR binding affinity and kinetics, we used a label-free biosensor assay in real-time. We measured the equilibrium dissociation constant (K^{D}) of TR14 for hTfR as 3.17×10⁻¹¹ mol/L, with 6.78×10³ _{M}⁻¹ₛ⁻¹ and 1.68×10⁻⁶ ₛ⁻¹ as the corresponding association (ka) and dissociation (kd) rate constants, respectively (FIG. 1D and Table 2).

To confirm the efficacy of the truncated aptamers for targeted delivery, we performed cell internalization assays using confocal microscopy on live cells. The two truncates containing 5′-GGG, TR14 S1 and TR14 S2, were internalized into HepG2 cells and PANC-1 (FIG. 2C). In contrast, the two truncates without 5′-GGG, TR14 ST1-1 and TR14 ST1-2, were not internalized into the cells (FIG. 2C). However, interestingly, the TR14 ST1-3 was internalized in both HepG2 cells and PANC-1 (FIG. 2C).

### Example 4—TR14 is Internalized into Cells Via Clathrin-Mediated Endocytosis and Competes with Transferrin, but not TfR Antibodies

To determine the mechanism of endocytosis, we utilized small molecules. In the pre-treatment of clathrin mediated endocytosis (CME) inhibitors such as chlorpromazine (CPZ), chloroquine (CQ), or dynasore, the uptake of TR14 was inhibited significantly (FIG. 1G). But, in the pre-treatment of clathrin independent endocytosis (CIE) inhibitors such as genistein (GEZ, caveolae/lipid mediated endocytosis inhibitor), or cytochalasin D (Cyto D, phagocytosis/micropinocytosis), the uptake was not interfered (FIG. 1G). With pre-incubation of TR14 on PANC-1 cells, internalized transferrin was significantly inhibited (FIG. 1H left). But, anti-transferrin receptor aptamer did not compete with transferrin receptor antibodies, suggesting that the binding site of TR14 and TfR antibodies might be different (FIG. 1H right).

### Example 5—TR14 is Distributed into Endosome and Lysosome Sub-Cellularly

To confirm subcellular co-localization of TR14, chemically synthesized Cy3 labelled TR14 was incubated on live cells where they expressed GFP fused to Rab5a (early endosome marker), Rab7a (late endosome marker), or Lamp1 (lysosomal marker). The co-localization was assessed on live cells with confocal microscopy with Airyscan. After 2 h incubation of TR14, we observed the multiple co-localizations of TR14 with early endosome, late endosome, and lysosome (FIG. 1I).

### Example 6—Truncated TR14 Aptamers Show Improved or Equivalent Binding Affinity Compared to the Parent TR14 Aptamer

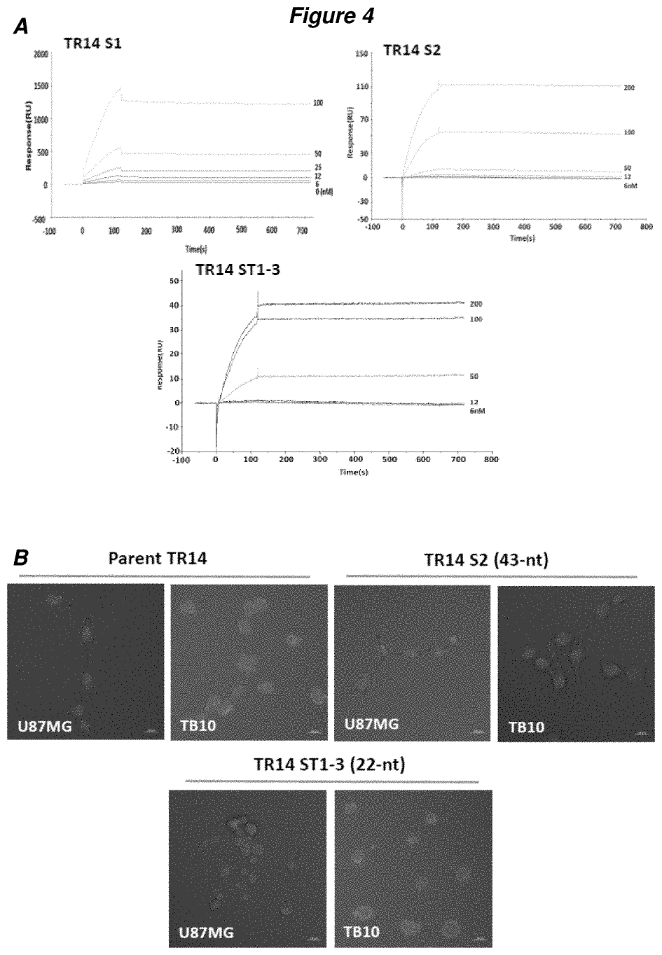

To measure binding affinity and kinetics of the truncated TR14 aptamers for hTfR1, we performed a label-free biosensor assay in real-time using a Biacore T100 instrument again. The resulting Biacore sensorgrams for TR14 S1, TR14 S2, and TR14 ST1-3 are presented in FIG. 4A; neither TR14 ST1-1 nor TR14 ST1-2 were measurable. Based on these results, we calculated equilibrium dissociation constants (K^{D}) and corresponding association (ka) and dissociation (kd) rate constants. As a result, the kinetics of TR14 S2 showed improved kinetic constants, TR14 ST1-3 remained the similar kinetic constants compared with parent TR14 (Table 2).

### Example 7—Truncated TR14-ST1-3 (22-Nt) Shows Cross-Reactivity to TfR2

To determine the potential cross-reactivity of the parent TR14, S2 (43-nt) and ST1-3 (22-nt) truncates, we tested chemically synthesized parent TR14, TR14-S3 or TR14-ST1-3 aptamer labelled with Cy3 on live cells imaging by confocal microscopy. To test this, we choose two glioblastoma cell lines (U87MG and TB10) that are selectively expressed of TfR1 or TfR2. The U87MG cells only express TfR1, not TfR2. In contrast, TB10 expresses TfR2, not TfR1. As a results, the parent TR14 bound to both TfR1 and TfR2. TR14-S2 showed selective binding to TfR1. TR14-ST1-3 showed cross-activity on both TfR1 and TfR2 (FIG. 4B).

### Example 8—TR14 Conjugated to C/EBPα-saRNA Demonstrates Anti-Proliferative Effects In Vitro

To achieve targeted delivery of C/EBPα-saRNA into pancreatic cancer cells, we constructed a conjugate that linked the TR14 aptamer with C/EBPα-saRNA (TR14-CEBPA). To maintain the functional integrity of the molecule, we placed a “sticky” sequence (a 16-nucleotide sequence that prevents structural hindrance) between TR14 and the C/EBPα-saRNA oligonucleotide.

To assess gene activation in vitro, we added TR14-CEBPA (100 nM) or IRRE-TR14-CEBPA (100 nM) (irrelevant/non-targeting aptamer control) to cultured PANC-1 cells, then used qPCR to measure mRNA expression of C/EBPα and its downstream target, p21. Cells treated with TR14-CEBPA showed significantly higher mRNA expression of C/EBPα and p21 (FIG. 3A) compared to IRRE control group. To measure the inhibition of cell proliferation by TR14-CEBPA, we performed MTS cell proliferation assays on PANC-1 cells treated with TR14-CEBPA or IRRE-TR14-CEBPA for 72 h. We also observed a significant reduction in cell proliferation following treatment with TR14-CEBPA compared to IRRE control group at the time point of 72 h (FIG. 3A). In treatment of conjugates of truncated TR14 (TR12-S3 or TR14 ST1-3) with C/EBPα-saRNA, we observed up-regulated mRNA expression of C/EBPα and its downstream target, p21, and inhibition of cancer cell proliferation by MTS assay (FIGS. 3B and E).

To increase pharmacokinetics of the conjugates in vivo, we chemically attached an albumin affinity tag to TR14 ST1-3 (termed tTR14). The tTR14 aptamers without or with an albumin affinity tag were named TC or TCT, respectively. We also made a TR14 ST1-3 with an affinity tag and a 10-uracil spacer (TCUT). A schematic illustration of these conjugates is depicted in FIG. 8; sequences are shown in Table 3. After treatment of PANC-1 cells with these conjugates, we also observed upregulation of C/EBPα and p21 (FIG. 3C) and inhibition of tumor cell proliferation (FIG. 3F), compared to IRRE control group.

### Example 9—TR14-CEBPA Shows Potent Anti-Tumor Effects in a Mouse Model of Advanced PDAC

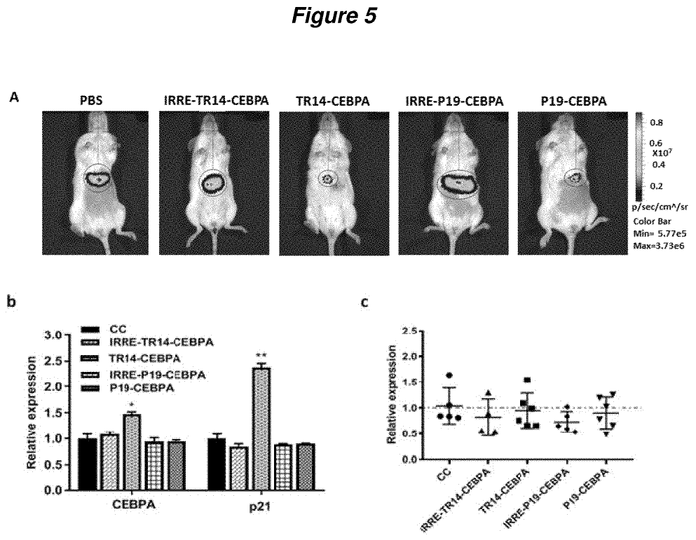

To determine the anti-tumor effects of the aptamer conjugates in advanced PDAC, we established a traceable animal model by implanting firefly luciferase-engineered PANC-1 cells (PANC-Luc) into the livers of NOD/SCID mice (i.e., intrahepatic pancreatic cancer cell implantation). In our previous studies, we demonstrated that P19-CEBPA showed potent anti-tumor effects in a local subcutaneous pancreatic cancer mouse model [30]. Therefore, in this study, we included P19-CEBPA in parallel with TR14-CEBPA to determine the anti-tumor efficacy in advanced PDAC model engrafted in liver of PANC-Luc. We randomly divided mice into five groups (n=5-6/group) and injected them with PBS, IRRE-TR14-CEBPA (1 nmol), TR14-CEBPA (1 nmol), IRRE-P19-CEBPA (1 nmol) or P19-CEBPA (1 nmol) via tail vein 3 times/week for 3 weeks. We monitored tumor growth by quantifying bioluminescence using an IVIS 200 in vivo imaging platform (FIG. 5A).

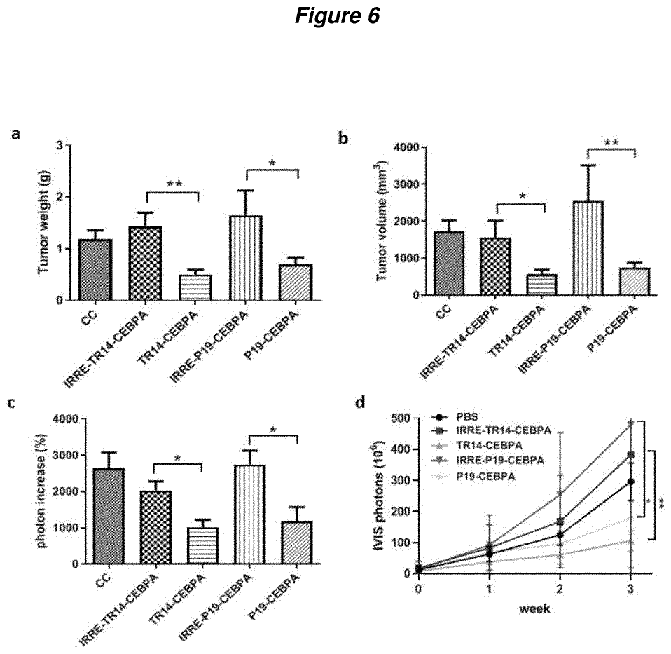

Given that hTFRC is also expressed in normal cells, we assessed TR14-CEBPA biodistribution indirectly by using qPCR to measure changes in mRNA expression of C/EBPα in hepatocytes isolated from liver sections of the treated mice. TR14-CEBPA-treated animals showed upregulation of C/EBPα and p21 mRNA levels in the liver, whereas P19-CEBPA-treated animals did not show the same effect (FIG. 5B). To investigate the extent to which the aptamer-conjugates affected liver function, we determined the expression level of albumin. Neither TR14-CEBPA nor P19-CEBPA induced any upregulation in albumin transcript mRNA (FIG. 5C). After treatment of mice with the aptamer-conjugates as described above, we assessed the burden of pancreatic cancer tumors on each implanted liver by measuring tumor weight and volume for three weeks. Both TR14-CEBPA and P19-CEBPA treatment groups showed significant reduction of tumor burden compared to the control groups (IRRE conjugates) (FIG. 6A and b). We subsequently quantified the bioluminescent signal, indicative of PANC-Luc tumor growth, which showed a significant inhibition of tumor growth following treatment with TR14-CEBPA and P19-CEBPA compared to controls (FIG. 6B). By tracing tumor growth using bioluminescence over time (0-3 weeks), we observed persistent anti-tumor effects following treatment with TR14-CEBPA and P19-CEBPA compared to controls in our model of advanced PDAC (FIG. 6D). In summary, our results showed significant reduction of advanced PDAC tumor burden in two groups; TR14-CEBPA and P19-CEBPA.

### Example 10—Targeted Delivery of C/EBPα-saRNA Conjugated with S2 or ST1-3 Increases the Expression of C/EBPα and its Downstream Effector p21 In Vitro

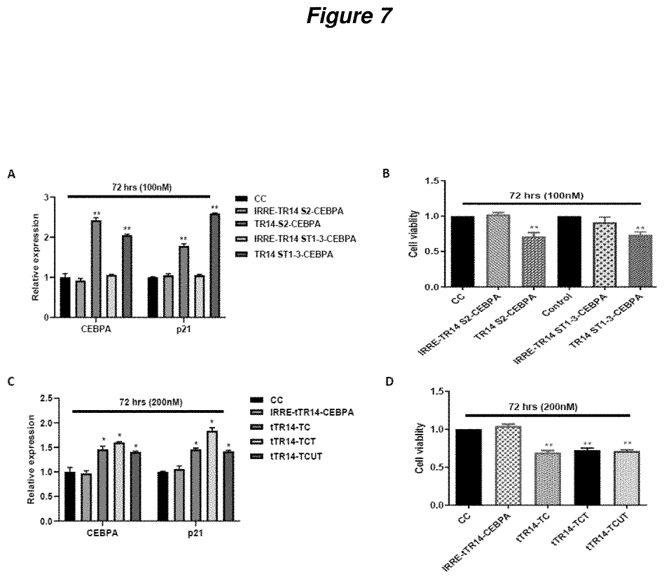

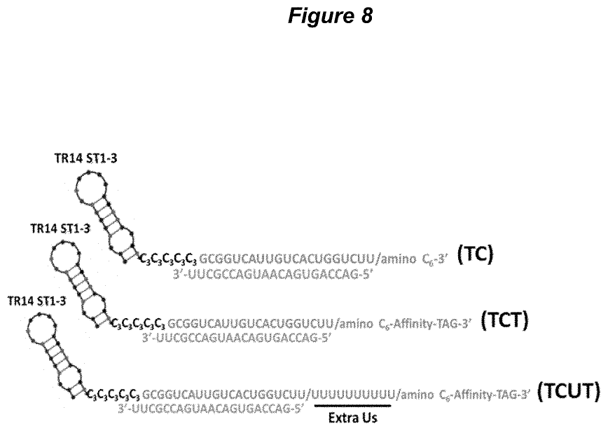

To determine the up-regulation of C/EBPα and p21 by TR14-S2-C/EBPα-saRNA and TR14-ST1-3-C/EBPα-saRNA, the conjugates were treated on PANC-1 cells. After treatment of conjugates, the upregulation of C/EBPα and p21 was observed (FIG. 7A). Also, these two conjugates inhibited the cell proliferation by MTS assay (FIG. 7B). To increase half-life of these conjugates in vivo, we chemically attached albumin affinity tag to TR14-ST1-3. Herein, TR14-ST1-3 is finalized truncated TR14 (tTR14). The tTR14 with or without albumin affinity tag was named TC, TCT or TCUT. The schematic illustration of these conjugates are depicted in FIG. 8. The detailed sequences are shown in Table 3. After treatment of these conjugates, we also observed the upregulation of C/EBPα and p21 (FIG. 7C). In MTS assay, these conjugates also inhibited the cancer cell proliferation (FIG. 7D).

### Example 11—the Truncated tTR14-CEBPA-saRNA-Albumin Affinity Tag (TC, TCT, TCUT) in Combination with Gemcitabine Shows Potent Anti-Tumor Effects in a Liver-Metastatic Pancreatic Mouse Model

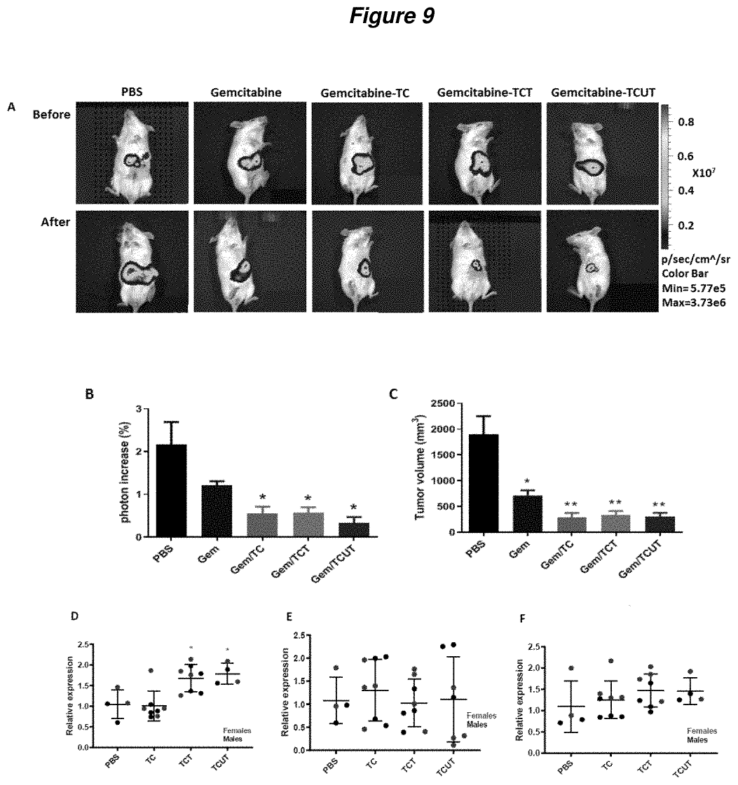

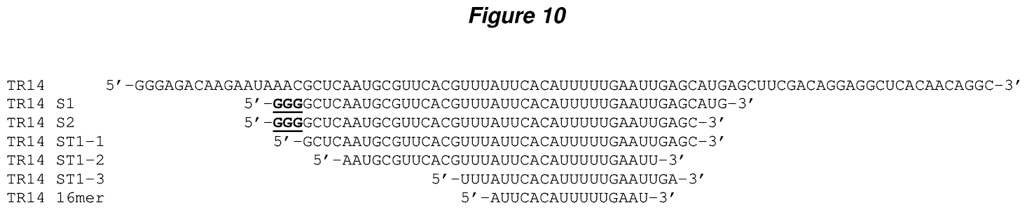

The current standard care for advanced PDAC is gemcitabine-based monotherapy or combinational therapies. Therefore, we tested the efficacy of albumin affinity-tagged tTR14-C/EBPα-saRNA conjugates (TC, TCT, TCUT; 1 nmol) in combination therapy with gemcitabine (50 mg/kg) in a mouse model of advanced PDAC. Representative bioluminescence images taken before and after treatment are shown in FIG. 9A. We quantitated anti-tumor effects by measuring tumor volume and change in photons. Compared with untreated control (PBS), all three conjugates (TC, TCT, TCUT) combined with gemcitabine caused a significant reduction of tumor burden in the advanced PDAC by measuring photon increase (FIGS. 9B and C). Compared to vehicle-treated control, our studies showed that gemcitabine alone reduced tumor volume ˜70%; the three conjugates (TC, TCT, TCUT) combined with gemcitabine reduced tumor growth up to ˜85% (FIG. 9B), suggesting that aptamer-C/EBPα-saRNAs might be used as adjuvant in combination with chemotherapy.

### Example 12—TCT and TCUT Increase the Transcript of C/EBPα in Liver Tissue

To test the efficacy of three conjugates (TC, TCT, TCUT) to deliver functionally active C/EBPα saRNA to liver metastatic PDAC, we ran an in vivo study on B6 mice, where an administration of 3 doses of 1 nmol via tail vein and tissue harvesting on day 8. Groups treated with TCT and TCUT conjugates showed increased the mRNA levels of C/EBPα, compared to untreated group (FIG. 9D). While no upregulation of mRNA levels of C/EBPα was observed in brain tissue (FIG. 9E). All groups showed none increased albumin mRNA levels after treatment (FIG. 9F).

### DISCUSSION

More than 75% of pancreatic cancer patients are diagnosed with metastatic advanced PDAC which show a dismal prognosis, and a 3% five-year overall survival rate (Loehrer et al, 2011). Although surgical resection of the primary tumor has been considered to improve the survival rate of PDAC patients, 85-90% are ineligible. Currently approved standard care of metastatic advanced PDAC is gemcitabine for systemic treatment. For advanced PDAC, the first-line chemotherapeutic options are irinotecan, combinational chemotherapies of gemcitabine and abraxane, or gemcitabine monotherapy. The second-line chemotherapeutics are combinational chemotherapies in gemcitabine with 5FU, Cisplatin or other small drugs (Ducreux et al, 2018). The anti-tumor effects of monotherapy are very disappointing, current trends of therapeutic options in PDAC are thriving combinational chemotherapeutics to improve the survival rate in advanced PDAC. Recent clinical trials into the combination of gemcitabine and nab-paclitaxel (Blomstand et al 2019) or triple combination of chemotherapy SOXIRI (S-1/Oxaliplatin/Irinotecan) (Akahori et al, 2019) show hematologic toxicity such as neutropenia, anemia, and leukopenia.

Because the expression of transcription factor C/EBPalpha is typically reduced during disease progression (Yoon et al 2016, Yamamoto et al 2014, Kumagai et al 2009, Timchenko et al 1996), it could be an effective target in aggressive advanced or metastatic pancreatic cancer. Currently, a challenge of oligonucleotide therapeutic field is delivery. Since the first FDA approval in 2004 for an aptamer-based treatment for neovascular age-related macular degeneration RNA aptamers have become very attractive therapeutic modalities.

With the aim of using them as delivery vehicles for saRNA treatment and to test the suitability of targeting C/EBPα for anti-cancer effects in aggressive metastatic PDAC, we employed two aptamers (TR14 and P19) which were conjugated with C/EBPα-saRNAs using sticky bridge sequences for targeted delivery. Both TR14-CEBPα and P19-CEBPα clearly demonstrated a significant increase in C/EBPα transcript and inhibition of cell proliferation in vitro.

To determine the efficacy of anti-tumor effects in vivo, herein, we established liver-metastatic pancreatic cancer model. Therefore, we implanted pancreatic cancer cells intrahepatically to determine the efficacy of therapeutics for metastatic PDAC. Using a mouse model of liver-metastasized pancreatic cancer, we compared the therapeutic efficacy of delivering C/EBPα-saRNA by two aptamers, TR14 and P19. We observed that both conjugates significantly inhibited tumor growth in a mouse model of liver-metastatic pancreatic cancer.

Analysis of gene expression in liver samples showed upregulation of C/EBPα in cells treated with TR14-C/EBPα. This result is not surprising, given that transferrin receptors are also expressed in normal cells. We did not observe any up-regulation of C/EBPα in the P19-CEBPα treatment group.

Herein we showed that TfR aptamers conjugated with C/EBPα-saRNA significantly reduced tumor burden in a liver-metastatic PDAC mouse model that was established for an advanced PDAC mouse model.

The known mechanism of action of gemcitabine is inhibition of DNA synthesis (Huang et al, 1991), and the anti-tumor effects of gemcitabine in pancreatic cancer have been previously determined. As the advanced PDAC is a main mortality of pancreatic cancer, we investigated the feasibility of gemcitabin in combination with other chemotherapeutics. To determine the feasibility of gemcitabine as for adjuvant chemotherapy in combination with C/EBPα-saRNA, C/EBPα-saRNA was targeted delivered into metastatic PDAC in treatment of gemcitabine in an advanced PDAC mouse model. In a result, the treatment of tTR14-C/EBPα-saRNA with gemcitabine adjuvant chemotherapy significantly reduced the tumor burden in liver-metastatic pancreatic cancer models, compared with mock.

The efficacy of C/EBPα-saRNA in combination with gemcitabine which can be used a regimen for adjuvant chemotherapy was determined in advanced PDAC herein by employing truncated TR14. To increase pharmacokinetics (PK) and pharmacodynamics (PD), an albumin affinity tag was chemically attached to the end of 3′ of aptamer. Without wishing to be bound by theory, we expect that it could alter the tissue biodistribution (PD) and pharmacokinetics (PK) profiles of aptamers.

In conclusion, we demonstrate that targeted delivery of CEBPα-saRNA by anti-hTFRC or pancreatic cancer-specific aptamers leads to potent anti-tumor effects in a mouse model of liver-metastatic pancreatic cancer. This suggests a therapeutic potential to cure patients with advanced pancreatic cancer. Given that the hTFRC is overexpressed in multiple cancer cells, hTFRC targeting will allow delivery of therapeutic payloads to other types of cancers. Furthermore, we showed that truncated TR14 aptamers delivered C/EBPα-saRNA into cancer cells. Finally, we demonstrated that gemcitabine can be used as adjuvant chemotherapy in combination with TR14 aptamers and, more specifically, TR14 aptamer-C/EBPα-saRNAs in advanced PDAC.

TABLE 1
Sequences of parent and truncates of transferrin receptor aptamers

| Name | Sequence |
|---|---|
| TR14 (Parent, 87-nt) | 5′- |
| | GGGAGACAAGAAUAAACGCUCAAUGCGUUCACGUUUAUUCACAUUUUU |
| | GAAUUGAGCAUGAGCUUCGACAGGAGGCUCACAACAGGC-3′ |
| TR14 S1 (46-nt) | 5′-GGGGCUCAAUGCGUUCACGUUUAUUCACAUUUUGAAUUGAGCAUG- |
| | 3′ |
| TR14 S2 (43-nt) | 5′-GGGGCUCAAUGCGUUCACGUUUAUUCACAUUUUUGAAUUGAGC-3′ |
| TR14 ST1-1 (40-nt) | 5′-GCUCAAUGCGUUCACGUUUAUUCACAUUUUUGAAUUGAGC-3′ |
| TR14 ST1-2 (32-nt) | 5′-AAUGCGUUCACGUUUAUUCACAUUUUUGAAUU-3′ |
| TR14 ST1-3 (22-nt) | 5′-UUUAUUCACAUUUUUGAAUUGA-3′ |

The truncated TFRC aptamer in bold. TR14 ST1-3 (22-nt) was used for in vivo assays.

TABLE 2
Measured association rate (ka), disassociation rate
(kd) and equilibrium dissociation constant (K_{D}).

| Name | ka (1/Ms) | kd (1/s) | K^{D} (M) | Remarks |
|---|---|---|---|---|
| TR14 (87-nt) | 6.78E+03 | 1.68E−06 | 3.17E−11 | |
| TR14 S1 (46-nt) | 1.30E+04 | 1.89E−05 | 1.46E−09 | ka and KD: |
| | | | | decreased. kd: |
| | | | | increased |
| TR14 S2 (43-nt) | 8.34E+08 | 5.77E−04 | 6.92E−13 | ka, kd and K_{D}: |
| | | | | increased |
| TR14 ST1-3 | 6.30E+06 | 1.38E−04 | 2.20E−11 | ka, kd and KD: |
| (22-nt) | | | | stayed in similar |
| | | | | range |

TABLE 3
Sequences of TR14 ST1-3 with CEBPA sense, CEBPA anti-sense, or affinity tag

| Name | Sequence |
|---|---|
| TR14 ST1-3 with | 5′-UUUAUUCACAUUUUUGAAUUGAoooooGCGGUCAUUGUCACUGGU |
| CEBPA sense (TC) | CUUX-3′ |
| TR14 ST1-3 with | 5′-UUUAUUCACAUUUUUGAAUUGAoooooGCGGUCAUUGUCACUGGU |
| CEBPA sense- | CUUX-Affinity TAG-3′ |
| Affinity TAG (TCT) | |
| TR14 ST1-3 with | 5′-UUUAUUCACAUUUUUGAAUUGAoooooGCGGUCAUUGUCACUGGU |
| CEBPA sense- | CUUUUUUUUUUUUX-Affinity-TAG-3 |
| Spacer-Affinity-TAG | |
| (TCUT) | |
| CEBPA anti-sense | 5′-GACCAGUGACAAUGACCGCUU-3′ |

### REFERENCES

A number of publications are cited above in order to more fully describe and disclose the invention and the state of the art to which the invention pertains. Full citations for these references are provided below. The entirety of each of these references is incorporated herein.
1. Huguet, F, Girard, N, Guerche, C S, Hennequin, C, Mornex, F, and Azria, D (2009). Chemoradiotherapy in the management of locally advanced pancreatic carcinoma: a qualitative systematic review. Journal of clinical oncology: official journal of the American Society of Clinical Oncology 27: 2269-2277.
2. Shaib, Y H, Davila, J A, and El-Serag, H B (2006). The epidemiology of pancreatic cancer in the United States: changes below the surface. Alimentary pharmacology & therapeutics 24: 87-94.
3. Houg, D S, and Bijlsma, M F (2018). The hepatic pre-metastatic niche in pancreatic ductal adenocarcinoma. Molecular cancer 17: 95.
4. Mohammad, A A (2018). Advanced pancreatic cancer: The standard of care and new opportunities. Oncology reviews 12: 370.
5. Paulson, A S, Tran Cao, H S, Tempero, M A, and Lowy, A M (2013). Therapeutic advances in pancreatic cancer. Gastroenterology 144: 1316-1326.
6. Ma, S J, Prezzano, K M, Hermann, G M, and Singh, A K (2018). Dose escalation of radiation therapy with or without induction chemotherapy for unresectable locally advanced pancreatic cancer. Radiation oncology 13: 214.
7. Rosenblum, D, Joshi, N, Tao, W, Karp, J M, and Peer, D (2018). Progress and challenges towards targeted delivery of cancer therapeutics. Nature communications 9: 1410.
8. Zuker, M (2003). Mfold web server for nucleic acid folding and hybridization prediction. Nucleic acids research 31: 3406-3415.
9. Zadeh, J N, Steenberg, C D, Bois, J S, Wolfe, B R, Pierce, M B, Khan, A R, et al. (2011). NUPACK: Analysis and design of nucleic acid systems. Journal of computational chemistry 32: 170-173.
10. Hernandez, F J, Kalra, N, Wengel, J, and Vester, B (2009). Aptamers as a model for functional evaluation of LNA and 2′-amino LNA. Bioorganic & medicinal chemistryletters 19: 6585-6587.
11. Soontornworajit, B, Zhou, J, Snipes, M P, Battig, M R, and Wang, Y (2011). Affinity hydrogels for controlled protein release using nucleic acid aptamers and complementary oligonucleotides. Biomaterials 32: 6839-6849
12. Yoon, S, Huang, K W, Reebye, V, Spalding, D, Przytycka, T M, Wang, Y, et al. (2017). Aptamer-Drug Conjugates of Active Metabolites of Nucleoside Analogs and Cytotoxic Agents Inhibit Pancreatic Tumor Cell Growth. Molecular therapy Nucleic acids 6: 80-88.
13. Yoon, S, Lee, G, Han, D, Song, J Y, Kang, K S, and Lee, Y S (2010). Neutralization of infectivity of porcine circovirus type 2 (PCV2) by capsid-binding 2′F-RNA aptamers. Antiviral research 88: 19-24.
14. Yoon, S, Huang, K W, Reebye, V, Mintz, P, Tien, Y W, Lai, H S, et al. (2016). Targeted Delivery of C/EBPalpha-saRNA by Pancreatic Ductal Adenocarcinoma-specific RNA Aptamers Inhibits Tumor Growth In Vivo. Molecular therapy: the journal of the American Society of Gene Therapy 24: 1106-1116.
15. Yamamoto, K, Tateishi, K, Kudo, Y, Sato, T, Yamamoto, S, Miyabayashi, K, et al. (2014). Loss of histone demethylase KDM6B enhances aggressiveness of pancreatic cancer through downregulation of C/EBPalpha. Carcinogenesis.
16. Kumagai, T, Akagi, T, Desmond, J C, Kawamata, N, Gery, S, Imai, Y, et al. (2009). Epigenetic regulation and molecular characterization of C/EBPalpha in pancreatic cancer cells. International journal of cancer Journal international du cancer 124: 827-833.
17. Timchenko, N A, Wilde, M, Nakanishi, M, Smith, J R, and Darlington, G J (1996). CCAAT/enhancer-binding protein alpha (C/EBP alpha) inhibits cell proliferation through the p21 (WAF-1/CIP-1/SDI-1) protein. Genes & development 10: 804-815.
18. Gragoudas, E S, Adamis, A P, Cunningham, E T, Jr., Feinsod, M, Guyer, D R, and Group, VISiONCT (2004). Pegaptanib for neovascular age-related macular degeneration. The New England journal of medicine 351: 2805-2816.
19. Loehrer, P J, Sr., Feng, Y, Cardenes, H, Wagner, L, Brell, J M, Celia, D, et al. (2011). Gemcitabine alone versus gemcitabine plus radiotherapy in patients with locally advanced pancreatic cancer: an Eastern Cooperative Oncology Group trial. Journal of clinical oncology: official journal of the American Society of Clinical Oncology 29: 4105-4112.
20. Ducreux, M, Seufferlein, T, Van Laethem, J L, Laurent-Puig, P, Smolenschi, C, Malka, D, et al. (2018). Systemic treatment of pancreatic cancer revisited. Semin Oncol.
21. Blomstrand, H, Scheibling, U, Bratthall, C, Green, H, and Elander, N O (2019). Real world evidence on gemcitabine and nab-paclitaxel combination chemotherapy in advanced pancreatic cancer. BMC Cancer 19: 40.
22. Akahori, T, Sho, M, Yanagimoto, H, Satoi, S, Nagai, M, Nishiwada, S, et al. (2019). Phase II Study of the Triple Combination Chemotherapy of SOXIRI (S-1/Oxaliplatin/Irinotecan) in Patients with Unresectable Pancreatic Ductal Adenocarcinoma. Oncologist.
23. Huang, P, Chubb, S, Hertel, L W, Grindey, G B, and Plunkett, W (1991). Action of 2′,2′-difluorodeoxycytidine on DNA synthesis. Cancer research 51: 6110-6117.
24. Tuli, R, Shiao, S L, Nissen, N, Tighiouart, M, Kim, S, Osipov, A, et al. (2019). A phase 1 study of veliparib, a PARP-1/2 inhibitor, with gemcitabine and radiotherapy in locally advanced pancreatic cancer. EBioMedicine.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

### Sequences

The aptamers TR14 and TR14 S2 are disclosed in WO2016/061386, whilst TR14 ST1-3 is disclosed in WO2019/033051.

| SEQ ID NO: | Name (length) | Sequence |
|---|---|---|
| 1 | TR14 (Parent, 87-nt) | 5′-GGGAGACAAGAAUAAACGCUCAAUGCGUUCACGUUUAU |
| | | UCACAUUUUUGAAUUGAGCAUGAGCUUCGACAGGAGGCU |
| | | CACAACAGGC-3′ |
| 2 | TR14 S1 (46-nt) | 5′-GGGGCUCAAUGCGUUCACGUUUAUUCACAUUUUUGAAU |
| | | UGAGCAUG-3′ |
| 3 | TR14 S2 (43-nt) | 5′-GGGGCUCAAUGCGUUCACGUUUAUUCACAUUUUUGAAU |
| | | UGA GC-3′ |
| 4 | TR14 ST1-1 (40-nt) | 5′-GCUCAAUGCGUUCACGUUUAUUCACAUUUUUGAAUUG |
| | | AGC-3′ |
| 5 | TR14 ST1-2 (32-nt) | 5′-AAUGCGUUCACGUUUAUUCACAUUUUUGAAUU-3′ |
| 6 | TR14 ST1-3 (22-nt) | 5′-UUUAUUCACAUUUUUGAAUUGA-3′ |
| 7 | SIRT1 saRNA sense | 5′-AUAUGUCCUCCUGGGAAGAUU-3′ |
| 8 | SIRT1 saRNA | 5′-UCUUCCCAGGAGGACAUAUUU-3′ |
| | antisense | |
| 9 | C/EBPα saRNA | 5′-GCGGUCAUUGUCACUGGUCUU-3′ |
| | sense | |
| 10 | C/EBPα saRNA | 5′-GACCAGUGACAAUGACCGCUU-3′ |
| | antisense | |
| 11 | P19 | 5′-GGGAGACAAGAAUAAACGCUCAAUGGCGAAUGCCCGCC |
| | | UAAUAGGGCGUUAUGACUUGUUGAGUUCGACAGGAGGCU |
| | | CACAACAGGC-3′ |
| 12 | T14 16mer truncation | 5-AUUCACAUUUUUGAAU-3′ |
| 13 | Human transferrin | MMDQARSAFSNLFGGEPLSYTRFSLARQVDGDNSHVEMKLA |
| | receptor protein 1 | VDEEENADNNTKANVTKPKRCSGSICYGTIAVIVFFLIGFMIGYL |
| | isoform 1 | GYCKGVEPKTECERLAGTESPVREEPGEDFPAARRLYWDDL |
| | (GI:189458817; | KRKLSEKLDSTDFTGTIKLLNENSYVPREAGSQKDENLALYVE |
| | NCBI Reference | NQFREFKLSKVWRDQHFVKIQVKDSAQNSVIIVDKNGRLVYLV |
| | Sequence: | ENPGGYVAYSKAATVTGKLVHANFGTKKDFEDLYTPVNGSIVI |
| | NP_003225.2) | VRAGKITFAEKVANAESLNAIGVLIYMDQTKFPIVNAELSFFGH |
| | (GI:189458816; | AHLGTGDPYTPGFPSFNHTQFPPSRSSGLPNIPVQTISRAAAE |
| | NCBI Reference | KLFGNMEGDCPSDWKTDSTCRMVTSESKNVKLTVSNVLKEIKI |
| | Sequence: | LNIFGVIKGFVEPDHYVVVGAQRDAWGPGAAKSGVGTALLLKL |
| | NM_003234.3 | AQMFSDMVLKDGFQPSRSIIFASWSAGDFGSVGATEWLEGYL |
| | (transcript variant 1)) | SSLHLKAFTYINLDKAVLGTSNFKVSASPLLYTLIEKTMQNVKH |
| | (GI:189458818; | PVTGQFLYQDSNWASKVEKLTLDNAAFPFLAYSGIPAVSFCFC |
| | NCBI Reference | EDTDYPYLGTTMDTYKELIERIPELNKVARAAAEVAGQFVIKLT |
| | Sequence: | HDVELNLDYERYNSQLLSFVRDLNQYRADIKEMGLSLQWLYS |
| | NM_001128148.2 | ARGDFFRATSRLTTDFGNAEKTDRFVMKKLNDRVMRVEYHFL |
| | (transcript variant 2) | SPYVSPKESPFRHVFWGSGSHTLPALLENLKLRKQNNGAFNE |
| | | TLFRNQLALATWTIQGAANALSGDVWDIDNEF |
| 14 | TR14 ST1-3 with | 5′-UUUAUUCACAUUUUUGAAUUGAOOOOOGCGGUCAUUGUC |
| | CEBPA sense | ACUGGUCUUX-3′ |
| 15 | TR14 ST1-3 with | 5′-UUUAUUCACAUUUUUGAAUUGAOOOOOGCGGUCAUUGUC |
| | CEBPA sense- | ACUGGUCUUX-Affinity TAG-3′ |
| | Affinity TAG | |
| 16 | TR14 ST1-3 with | 5′-UUUAUUCACAUUUUUGAAUUGAOOOOOGCGGUCAUUGUC |
| | CEBPA sense- | ACUGGUCUUUUUUUUUUUUX-Affinity-TAG-3 |
| | Spacer-Affinity-TAG | |

1. (canceled)

2. (canceled)

3. A method of treating or preventing a disease or disorder in a subject in need thereof, the method comprising administering simultaneously or sequentially to the subject an effective amount of a nucleic acid compound and an effective amount of an inhibitor of DNA synthesis, wherein said nucleic acid compound comprises, or consists of a nucleic acid sequence capable of binding to a transferrin receptor (TfR).

4. The method according to claim 3, wherein said nucleic acid sequence has at least 95% sequence identity to a sequence selected from SEQ ID NOs: 1 to 6.

5. (canceled)

6. The method according to claim 3, wherein the nucleic acid sequence has a length of 22 nucleotides.

7. The method according to claim 3, wherein the inhibitor of DNA synthesis is a nucleoside analogue, and wherein the nucleoside analogue is gemcitabine.

8. (canceled)

9. The method according to claim 3, wherein the disease or disorder is cancer.

10. The method according to claim 9, wherein the cancer is pancreatic cancer.

11. The method according to claim 3, wherein the nucleic acid compound further comprises a moiety attached to said nucleic acid sequence, wherein said moiety is a therapeutic moiety.

12. (canceled)

13. The method according to claim 11, wherein said therapeutic moiety is covalently attached to said nucleic acid sequence.

14. The method according to claim 13, wherein said therapeutic moiety is an anticancer therapeutic moiety, and wherein said therapeutic moiety is a C/EBPalpha saRNA moiety, a SIRT1 saRNA moiety, or a HNF saRNA moiety.

15. (canceled)

16. A nucleic acid compound comprising, or consisting of, a nucleic acid sequence having at least 95% sequence identity to SEQ ID NO: 5, wherein said nucleic acid sequence is at least 30 nucleotides in length and at most 50 nucleotides in length, and wherein the nucleic acid sequence is capable of binding to a transferrin receptor (TfR).

17. The nucleic acid compound according to claim 16, wherein said nucleic acid sequence comprises or consists of a sequence having at least 95% sequence identity to SEQ ID NO: 2 or 4.

18. (canceled)

19. (canceled)

20. (canceled)

21. (canceled)

22. (canceled)

23. (canceled)

24. The nucleic acid compound according to claim 16, wherein the nucleic acid sequence is capable of binding to both TfR1 and TfR2.

25. The nucleic acid compound according to claim 16, further comprising a moiety attached to said nucleic acid sequence, wherein said moiety is a therapeutic moiety.

26. (canceled)

27. (canceled)

28. (canceled)

29. The nucleic acid compound according to claim 25, wherein said moiety is a C/EBPalpha saRNA moiety, a SIRT1 saRNA moiety, or a HNF saRNA moiety.

30. (canceled)

31. A pharmaceutical composition comprising a nucleic acid compound according to claim 16, optionally comprising a pharmaceutically acceptable excipient.

32. A method of delivering a compound moiety into a cell, the method comprising:
a. contacting a cell with the nucleic acid compound according to claim 16; and
b. allowing said nucleic acid compound to bind to a transferrin receptor on said cell and pass into said cell thereby delivering said compound moiety into said cell.

33. (canceled)

34. (canceled)

35. (canceled)

36. A method of treating or preventing a disease or disorder, the method comprising administering to a subject in need thereof an effective amount of a nucleic acid compound according to claim 16.

37. The method according to claim 36, wherein the disease or disorder is cancer, and wherein the method further comprises administering to the subject an anticancer agent.

38. (canceled)


SEQUENCE LISTING (19 sequences)

SEQ ID NO: 1 - RNA, 87 nt, Artificial Sequence
  TR14 (Parent, 87 nt)
gggagacaagaauaaacgcucaaugcguucacguuuauucacauuuuugaauugagcaugagcuucgacaggaggcucacaacaggc

SEQ ID NO: 2 - RNA, 46 nt, Artificial Sequence
  TR14 S1 (46 nt)
ggggcucaaugcguucacguuuauucacauuuuugaauugagcaug

SEQ ID NO: 3 - RNA, 43 nt, Artificial Sequence
  TR14 S2 (43 nt)
ggggcucaaugcguucacguuuauucacauuuuugaauugagc

SEQ ID NO: 4 - RNA, 40 nt, Artificial Sequence
  TR14 ST1-1 (40 nt)
gcucaaugcguucacguuuauucacauuuuugaauugagc

SEQ ID NO: 5 - RNA, 32 nt, Artificial Sequence
  TR14 ST1-2 (32 nt)
aaugcguucacguuuauucacauuuuugaauu

SEQ ID NO: 6 - RNA, 22 nt, Artificial Sequence
  TR14 ST1-3 (22-nt)
uuuauucacauuuuugaauuga

SEQ ID NO: 7 - RNA, 21 nt, Artificial Sequence
  SIRT1 saRNA sense
auauguccuccugggaagauu

SEQ ID NO: 8 - RNA, 21 nt, Artificial Sequence
  SIRT1 saRNA antisense
ucuucccaggaggacauauuu

SEQ ID NO: 9 - RNA, 21 nt, Artificial Sequence
  C/EBP? saRNA sense
gcggucauugucacuggucuu

SEQ ID NO: 10 - RNA, 21 nt, Artificial Sequence
  C/EBP? saRNA antisense
gaccagugacaaugaccgcuu

SEQ ID NO: 11 - RNA, 87 nt, Artificial Sequence
  P19
gggagacaagaauaaacgcucaauggcgaaugcccgccuaauagggcguuaugacuuguugaguucgacaggaggcucacaacaggc

SEQ ID NO: 12 - RNA, 16 nt, Artificial Sequence
  T14 16mer truncation
auucacauuuuugaau

SEQ ID NO: 13 - protein, 760 aa, Homo sapiens
  Transferrin receptor protein 1 isoform 1
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 14 - RNA, 44 nt, Artificial Sequence
  TR14 ST1-3 with CEBPA sense
  misc_feature (44)..(44): n is a, c, g, or u
uuuauucacauuuuugaauugagcggucauugucacuggucuun

SEQ ID NO: 15 - RNA, 44 nt, Artificial Sequence; identical to SEQ ID NO: 14
  TR14 ST1-3 with CEBPA sense-Affinity TAG
  misc_feature (44)..(44): n is a, c, g, or u

SEQ ID NO: 16 - RNA, 54 nt, Artificial Sequence
  TR14 ST1-3 with  CEBPA sense- Spacer-Affinity- TAG
  misc_feature (54)..(54): n is a, c, g, or u
uuuauucacauuuuugaauugagcggucauugucacuggucuuuuuuuuuuuun

SEQ ID NO: 17 - DNA, 23 bp, Artificial Sequence
  T7 RNA polymerase promoter region
taatacgactcactatagggaga

SEQ ID NO: 18 - DNA, 20 bp, Artificial Sequence
  T7 RNA polymerase promoter
taatacgactcactataggg

SEQ ID NO: 19 - RNA, 31 nt, Artificial Sequence
  TR14 ST1-3
gcggucauugucacuggucuuuuuuuuuuuu